SEQUENCE LISTING (3 sequences)

SEQ ID NO: 1 - protein, 4 aa, Homo sapiens
IETD

SEQ ID NO: 2 - DNA, 20 bp, Homo sapiens
gacttcagcagacatcctac

SEQ ID NO: 3 - DNA, 18 bp, artificial
  nonsense oligonucleotide
tggatccgacatgtcaga

(19) United States
(12) Patent Application Publication
(10) Pub. No.: US 2005/0208151 A1
(43) Pub. Date: Sep. 22, 2005

(54) Treatment of rheumatoid arthritis with FLIP antagonists

(75) Inventors: Vincent Jacques Hurez, Albany, CA (US); Saroja Ramanujan, Daly City, CA (US); Lisl K.M. Shoda, Menlo Park, CA (US); Leif Gustaf Wennerberg, Mountain View, CA (US); Seth G. Michelson, San Jose, CA (US); Nadine Defranoux, San Francisco, CA (US)

Correspondence Address: FISH & RICHARDSON P.C., PO BOX 1022, MINNEAPOLIS, MN, 55440-1022, US

(73) Assignee: Entelos, Inc., Foster City, CA (US)

(21) Appl. No.: 10/980,145

(22) Filed: Nov. 1, 2004

Related U.S. Application Data
(60) Provisional application No. 60/516,560, filed on Oct. 30, 2003.

(51) Int. Cl. A61K 38/17; A61K 48/00; A61K 31/277; A61K 31/7072; A61K 31/704; A61K 31/407; A61K 31/16; A61K 33/24; A61K 31/19

(52) U.S. Cl. 424/649; 514/12; 514/44; 514/34; 514/521; 514/49; 514/557; 514/410; 514/615

(57) ABSTRACT

The invention encompasses novel methods of treating rheumatoid arthritis and its symptoms and novel methods of identifying and screening for drugs useful in the treatment of rheumatoid arthritis and its clinical symptoms. Targeted manipulation of a computer model of a human rheumatic joint provided the surprising result that decreasing the activity of FLIP, an inhibitor of apoptosis, by at least 25% has a significant impact on the pathophysiology of rheumatoid arthritis. Inhibition of the activity of FLIP by at least 25% is predicted to alleviate the symptoms of rheumatoid arthritis.

### I. INTRODUCTION

### A. RELATED APPLICATIONS

This application claims the benefit of U.S. Provisional Application No. 60/516,560, filed Oct. 30, 2003, which is herein incorporated by reference.

### B. FIELD OF THE INVENTION

This invention relates to novel methods of treating rheumatoid arthritis and methods of identifying compounds useful in treating rheumatoid arthritis.

### C. BACKGROUND OF THE INVENTION

There are more than 100 forms of arthritis and of them, rheumatoid arthritis is the most painful and crippling form. Rheumatoid arthritis, a common disease of the joints, is an autoimmune disease that affects over 2 million Americans, with a significantly higher occurrence among women than men. In rheumatoid arthritis, the membranes or tissues (synovial membranes) lining the joints become inflamed (synovitis). Over time, the inflammation may destroy the joint tissues, leading to disability. Because rheumatoid arthritis can affect multiple organs of the body, rheumatoid arthritis is referred to as a systemic illness and is sometimes called rheumatoid disease. The onset of rheumatoid disease is usually in middle age, but frequently occurs in one's 20s and 30s.

The pain and whole-body (systemic) symptoms associated with rheumatoid disease can be disabling. Over time, rheumatoid arthritis can cause significant joint destruction, leading to deformity and difficulty with daily activities. It is not uncommon for people with rheumatoid arthritis to suffer from some degree of depression, which may be caused by pain and progressive disability. A study reports that one-fourth of people with rheumatoid arthritis are unable to work by 6 to 7 years after their diagnosis, and half are not able to work after 20 years (O'Dell, Rheumatoid arthritis: The clinical picture. In W J Koopman, ed., Arthritis and Allied Conditions: A Textbook of Rheumatology, 14th ed., vol. 1, chap. 58, pp. 1153-1186. Philadelphia: Lippincott Williams and Wilkins (2001)). Musculoskeletal conditions such as rheumatoid arthritis cost the U.S. economy nearly $65 billion per year in medical care and indirect expenses such as lost wages and production.

Synovial inflammation, rapid degradation of cartilage, and erosion of bone in affected joints are characteristic of rheumatoid arthritis (RA). Recent evidence indicates that skeletal tissue degradation and inflammation are regulated through overlapping but not identical biological processes in the rheumatoid joint and that therapeutic effects on these two aspects need not be correlated. Due to the complexity of the biological processes in the joint, mathematical and computer models can be used to help better understand the interactions between the various tissue compartments, cell types, mediators, and other factors involved in joint disease and healthy homeostasis. Several researchers have constructed simple models of the mechanical environment of the joint, rather than the biological processes of rheumatoid arthritis, and compared the results to patterns of disease and development in cartilage and bone (Wynarsky & Greenwald, J. Biomech., 16:241-251, 1983; Pollatschek & Nahir, J. Theor. Biol., 143:497-505, 1990; Beaupre et al., J. Rehabil. Res. Dev., 37:145-151, 2000; Shi et al., Acta Med. Okayama, 17:646-653, 1999). A computer manipulable mathematical model of joint diseases that includes multiple compartments including the synovial membrane and the interactions of these compartments is described in published PCT application WO 02/097706, published 5 Dec. 2002 and U.S. patent application Ser. No. 10/154,123, published 24 Apr. 2003 as 2003-0078759. Both publications are incorporated herein by reference in their entirety.

Rheumatoid arthritis is a chronic disease that, at present, can be controlled but not cured. The goal of treatment is relief of symptoms and keeping the disease from getting worse. The goals of most treatments for rheumatoid arthritis are to relieve pain, reduce inflammation, slow or stop the progression of joint damage, and improve a person's ability to function. Current approaches to treatment include lifestyle changes, medication, surgery, and routine monitoring and care. Medications used for the treatment of rheumatoid arthritis can be divided into two groups based on how they affect the progression of the disease: (1) symptom-relieving drugs and (2) disease-modifying drugs.

Medications to relieve symptoms, such as pain, stiffness, and swelling, may be used. Nonsteroidal anti-inflammatory drugs (NSAIDs), such as aspirin, ibuprofen, and naproxen are used to control pain and may help reduce inflammation. They do not control the disease or stop the disease from getting worse. Corticosteroids, such as prednisone and methylprednisolone (Medrol), are used to control pain and reduce inflammation. They may control the disease or stop the disease from getting worse; however, using corticosteroids as the only therapy for an extended time is not considered the best treatment. Corticosteroids are often used to control symptoms and flares of joint inflammation until anti-rheumatic drugs reach their full effectiveness, which can take up, to 6 months. Nonprescription medications such as acetaminophen and topical medications such as capsaicin are used to control pain, but do not usually affect joint swelling or worsening of the disease.

Disease-modifying anti-rheumatic drugs (DMARDs) are used to control the progression of rheumatoid arthritis and to try to prevent joint deterioration and disability. These anti-rheumatic drugs are often given in combination with other anti-rheumatic drugs or with other medications, such as nonsteroidal anti-inflammatory drugs. Disease-modifying anti-rheumatic drugs commonly prescribed for rheumatoid arthritis include antimalarial medications such as hydroxycholoroquine (Plaquenil) or chloroquine (Aralen), methotrexate (e.g., Rheumatrex), sulfasalazine (Azulfidine), leflunomide (Arava), etanercept (Enbrel), infliximab (Remicade), adalimumab (Humira) and anakinra (Kineret). DMARDs less commonly prescribed for rheumatoid arthritis include azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). Some of these anti-rheumatic drugs can take up to 6 months to work. Many have serious side effects.

Thus a need exists for new, therapeutically effective drugs for the treatment of rheumatoid arthritis as well as new methods for identifying such drugs.

### D. SUMMARY OF THE INVENTION

In one aspect, the invention provides methods for alleviating at least one symptom of rheumatoid arthritis comprising administering a therapeutically effective amount of an antagonist of FLIP (FLICE-Inhibitory Protein) activity to a patient having rheumatoid arthritis, wherein the antagonist decreases FLIP activity by at least 25%. Preferably, the antagonist will decrease FLIP activity by at least 50%. More preferably, the antagonist decreases FLIP activity by at least 75%. Most preferably, the antagonist decreases FLIP activity by at least 95%. In preferred embodiments, the patient is a methotrexate resistant patient, a TNF-α blockade cartilage nonresponder (CNR), a TNF-α blockade hyperplasia nonresponder (HNR), or a TNF-α blockade double nonresponder (DNR). The antagonist of FLIP activity may be a protein, nucleic acid or small molecule inhibitor. A “small molecule” is defined herein as a molecule having a molecular weight of less than 1000 daltons. Preferred protein antagonists include, but are not limited to oxidized low-density lipoprotein, ectopic-p53, IFN-β, PPAR ligand, E1A, and hemin. Preferred small molecule inhibitors include, but are not limited to, cyclohexamide, actinomycin D, 5-fluorouracil, doxorubicin, cisplatin, sodium butyrate, bisindolylmaleimides, H7, calphostin C, chelerythrine chloride, CDDO (triterpenoid 2-cyano-3,12-dioxoolean-1,9-dien-28-oic acid) and PS-341.

In another aspect, the invention provides methods for decreasing density of synovial cells in a joint comprising administering a therapeutically effective amount of an antagonist of FLIP activity to a patient having a condition associated with abnormally increased synovial cell density, wherein antagonist decreases FLIP activity by at least 25%. Preferably, the antagonist will decrease FLIP activity by at least 50%. More preferably, the antagonist decreases FLIP activity by at least 75%. Most preferably, the antagonist decreases FLIP activity by at least 95%.

In yet another aspect, the invention provides methods for decreasing cartilage degradation in a joint comprising administering a therapeutically-effective amount of an antagonist of FLIP activity to a patient having a condition associated with an abnormally high rate of cartilage degradation, wherein the antagonist decreases FLIP activity by at least 25%. Preferably, the antagonist will decrease FLIP activity by at least 50%. More preferably, the antagonist decreases FLIP activity by at least 75%. Most preferably, the antagonist decreases FLIP activity by at least 95%.

Another aspect of the invention provides methods of decreasing bone erosion in a joint comprising administering a therapeutically effective amount of an antagonist of FLIP activity to a patient having a condition associated with an abnormally high rate of bone erosion, wherein the antagonist decreases FLIP activity by at least 25%. Preferably, the antagonist will decrease FLIP activity by at least 50%. More preferably, the antagonist decreases FLIP activity by at least 75%. Most preferably, the antagonist decreases FLIP activity by at least 95%.

In another aspect, the invention provides methods of alleviating at least one symptom of an inflammatory disease comprising administering a therapeutically effective amount of an antagonist of FLIP activity to a patient having an inflammatory disease, wherein the antagonist decreases FLIP activity by at least 25%. Preferably, the antagonist will decrease FLIP activity by at least 50%. More preferably, the antagonist decreases FLIP activity by at least 75%. Most preferably, the antagonist decreases FLIP activity by at least 95%. In preferred embodiments, the inflammatory disease is selected from the group consisting of diabetes, arteriosclerosis, inflammatory aortic aneurysm, restenosis, ischemia/reperfusion injury, glomerulonephritis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. More preferably the inflammatory disease is an arthritis, such as rheumatoid arthritis, psoratic arthritis, coxarthritis, osteoarthritis, or polyarthritis. Most preferably, the inflammatory disease is rheumatoid arthritis.

Yet another aspect of the invention provides methods of alleviating at least one symptom of rheumatoid arthritis, comprising administering an antagonist of FLIP activity and a disease-modifying anti-rheumatic drug to a patient having rheumatoid arthritis. The disease-modifying anti-rheumatic drug can be any drug that, in combination with FLIP antagonism, provides a better clinical outcome than treatment with FLIP antagonism or the anti-rheumatic drug alone. Exemplary disease-modifying anti-rheumatic drugs include hydroxycholoroquine (Plaquenil), chloroquine (Aralen), methotrexate (e.g., Rheumatrex), sulfasalazine (Azulfidine), leflunomide (Arava), etanercept (Enbrel), infliximab (Remicade), adalimumab (Humira), anakinra (Kineret), azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). In preferred embodiments, the anti-rheumatic drug is methotrexate, an interleukin-1 receptor antagonist, such as Anakinra, or a steroid, such as methylprednisolone.

A different aspect of the invention provides methods of manufacturing a drug for use in the treatment of rheumatoid arthritis comprising identifying a compound as useful in the treatment of rheumatoid arthritis by (i) comparing an amount of FLIP activity in the presence of the compound with an amount of FLIP activity in the absence of the compound and (ii) identifying the compound as useful in the treatment of rheumatoid arthritis when the amount of FLIP activity in the presence of the compound is at least 25% lower than the amount of FLIP activity in the absence of the compound. The compound is then formulated for human consumption. Preferably, the compound will decrease FLIP activity by at least 50%. More preferably, the compound decreases FLIP activity by at least 75%. Most preferably, the compound decreases FLIP activity by at least 95%.

Another aspect of the invention provides methods for screening a collection of compounds for a compound useful in the treatment of rheumatoid arthritis comprising, (a) comparing an amount of FLIP activity in the presence of the compound with an amount FLIP activity in the absence of the compound; and (b) selecting the compound as useful in the treatment of rheumatoid arthritis when the amount of FLIP activity in presence the of the compound is at least 25% lower than the amount of FLIP activity in the absence of the compound. Preferably, the compound will decrease FLIP activity by at least 50%. More preferably, the compound decreases FLIP activity by at least 75%. Most preferably, the compound decreases FLIP activity by 95%. In one embodiment of the invention, steps (a) and (b) are repeated for each compound of the collection, and at least one compound of the collection is selected as useful in the treatment of rheumatoid arthritis.

One embodiment encompasses measuring the amount of FLIP activity by a process comprising the steps of adding a caspase-8 substrate to a cell lysate in the presence or absence of the compound, and measuring the amount of caspase-8 substrate cleaved, wherein the compound is identified as useful in the treatment of rheumatoid arthritis when the amount of caspase-8 substrate cleaved in the presence of the compound is at least 50% higher than the amount of caspase-8 substrate cleaved in the absence of the compound. More preferably, the compound is identified when the amount of caspase-8 substrate cleaved is at least 100% higher in the presence of the compound than the absence of the compound. Most preferably, the compound is identified when the amount of caspase-8 substrate cleaved is at least 200% higher in the presence of the compound than the absence of the compound. Alternatively, the amount of FLIP activity is measured by determining the amount of FLIP protein expressed in the presence and absence of the compound. Optionally the cells may be exposed to an inducer of apoptosis in the presence or absence the compound prior to determining the amount of FLIP activity.

In yet another aspect, the invention provides methods of manufacturing a drug for use in the treatment of rheumatoid arthritis comprising identifying a compound as useful in the treatment of rheumatoid arthritis by (i) comparing an amount of macrophage apoptosis in the presence of the compound with an amount macrophage apoptosis in the absence of the compound, and (ii) identifying the compound as useful in the treatment of rheumatoid arthritis when the amount of macrophage apoptosis in the presence of the compound is at least 50% greater than the amount of macrophage apoptosis in the absence of the compound. The identified compound is then formulated for human consumption. More preferably, the compound is identified as useful in the treatment of rheumatoid arthritis when the amount of macrophage apoptosis in the presence of the compound is at least 100% greater than the amount of macrophage apoptosis in the absence of the compound. Most preferably, the compound is identified as useful in the treatment of rheumatoid arthritis when the amount of macrophage apoptosis in the presence of the compound is at least 200% greater than the amount of macrophage apoptosis in the absence of the compound. In a desired embodiment, the identified compound decreases FLIP activity by 25%, more preferably by 50%, even more preferably by 70% and most preferably by 95%.

The invention also provides methods of screening a collection of compounds for a compound useful in the treatment of rheumatoid arthritis comprising comparing an amount of macrophage apoptosis in the presence of the compound with an amount of macrophage apoptosis in the absence of the compound, and selecting the compound as useful in the treatment of rheumatoid arthritis when the amount of macrophage apoptosis in the presence of the compound is at least 50% greater than the amount of macrophage apoptosis in the absence of the compound. More preferably, the compound is selected when the amount of macrophage apoptosis in the presence of the compound is at least 100% greater than the amount of macrophage apoptosis in the absence of the compound. Most preferably, the compound is selected when the amount of macrophage apoptosis in the presence of the compound is at least 200% greater than the amount of macrophage apoptosis in the absence of the compound. In one embodiment of the invention, steps (a) and (b) are repeated for each compound of the collection, and at least one compound of the collection is selected as useful in the treatment of rheumatoid arthritis.

The amount of macrophage apoptosis may be determined by any apoptosis measurement technique, now known or discovered in the future. One embodiment of the invention measures the amount of macrophage apoptosis by a process comprising the steps of exposing a population of cells to an inducer of apoptosis in the presence or absence of the compound, and measuring the percentage of cells having DNA fragmentation, wherein the percentage of cells having DNA fragmentation represents the amount of macrophage apoptosis. The percentage of cells having DNA fragmentation may be measured by any method know in the art, including propidium iodide uptake or TUNEL (terminal deoxynucleotidyl transferase-mediated 2′-deoxyuridine 5′-triphosphate-biotin nick-end labeling) assay. In yet another embodiment of the invention, the amount of macrophage apoptosis is measured by a process comprising the steps of exposing a population of cells to an inducer of apoptosis in the presence or absence of the compound, and measuring the percentage of cells expressing phosphatidylserine on the extracellular surface of the cell membrane, wherein the percentage of cells expressing phosphatidylserine on the extracellular surface of the cell membrane represents the amount of macrophage apoptosis. Preferably the expression of phosphatidylserine on the extracellular surface of the cytoplasmic membrane is measured by binding of annexin V to the phosphatidylserine.

An aspect of the invention provides methods of identifying a compound useful for treatment of an inflammatory disease comprising (a) comparing an amount of FLIP activity in the presence of the compound with an amount of FLIP activity in the absence of the compound; and (b) identifying the compound as useful for treatment of an inflammatory disease when the amount of FLIP activity in the presence of the compound is lower than the amount of FLIP activity in the absence of the compound.

It will be appreciated by one of skill in the art that the embodiments summarized above may be used together in any suitable combination to generate additional embodiments not expressly recited above, and that such embodiments are considered to be part of the present invention

### II. BRIEF DESCRIPTION OF THE FIGURES

For a better understanding of the nature and objects of some embodiments of the invention, reference should be made to the following detailed description taken in conjunction with the accompanying drawings, in which:

FIG. 1 demonstrates the effect of FLIP blockade on synovial cell density.

FIG. 2 demonstrates the effect of FLIP blockade on cartilage degradation.

FIG. 3 provides the relative contribution of macrophage apoptosis (max cellular protection), T-cell apoptosis and T-cell production of IL-2 to the effect of FLIP blockade on the clinical outcomes in the reference virtual patient.

FIG. 4A illustrates the relative contribution of macrophage apoptosis (max cellular protection), T-cell apoptosis and T-cell production of IL-2 to the clinical outcomes in a methotrexate resistant patient utilizing the most likely maximum effect of FLIP blockade. FIG. 4B illustrates the relative contribution of macrophage apoptosis (max cellular protection), T-cell apoptosis and T-cell production of IL-2 on the global effect in a methotrexate resistant patient utilizing the upper maximum effect of FLIP blockade.

FIG. 5 demonstrates the effect of FLIP blockade on synovial cell density in a methotrexate resistant patient.

FIG. 6 demonstrates the effect of FLIP blockade on cartilage degradation in a methotrexate resistant patient

FIG. 7A provides a comparison of FLIP inhibition with expected increase in macrophage apoptosis in the RA reference patient. FIG. 7B provides a comparison of macrophage apoptosis levels with the therapeutic indexes of synovial cell density and cartilage degradation.

FIG. 8A provides a comparison of FLIP inhibition with expected increase in macrophage apoptosis for a methotrexate-resistant patient. FIG. 8B provides a comparison of macrophage apoptosis levels with the therapeutic indexes of synovial cell density and cartilage degradation in a methotrexate-resistant patient.

FIG. 9 illustrates the biochemistry of apoptosis.

FIG. 10 provides a diagram of the structure of FLIP and caspase-8.

FIG. 11 illustrates the results of simulating the effects of FLIP inhibition in combination with other anti-rheumatic drugs on synovial cell density in a rheumatoid arthritis patient.

FIG. 12 illustrates the results of simulating the effects of FLIP inhibition in combination with other anti-rheumatic drugs on cartilage degradation rates in a rheumatoid arthritis patient.

FIG. 13 illustrates the results of simulating the effects of FLIP inhibition in combination with other anti-rheumatics drug on synovial cell density in a methotrexate nonresponder.

FIG. 14 illustrates the results of simulating the effects of FLIP inhibition in combination with other anti-rheumatic drugs on cartilage degradation rates in a methotrexate nonresponder.

FIG. 15 illustrates the results of simulating the effects of FLIP inhibition in combination with other anti-rheumatic drugs on synovial cell density in a TNF-α blockade hyperplasia nonresponder.

FIG. 16 illustrates the results of simulating the effects of FLIP inhibition in combination with other anti-rheumatic drugs on cartilage degradation rates in a TNF-α blockade hyperplasia nonresponder.

### III. DETAILED DESCRIPTION

A. Overview

In general this invention can be viewed as encompassing a novel method of treating inflammatory disease, such as rheumatoid arthritis, and novel methods of identifying and screening for drugs useful in the treatment of inflammatory diseases and their clinical symptoms. Through the use of a computer model of a human rheumatic joint, the inventors have made the discovery that the activity of FLIP, an inhibitor of apoptosis, particularly macrophage apoptosis, has a significant impact on the pathophysiology of rheumatoid arthritis. Inhibition of the activity of FLIP is predicted to alleviate the symptoms of inflammatory diseases, such as rheumatoid arthritis.

B. Definitions

The term “abnormally high rate of cartilage degradation,” as used herein, refers to a detectable joint space narrowing as determined by standard radiographic measures. In a non-diseased joint, narrowing is not detectable.

The term “abnormally high rate of bone erosion,” as used herein, refers to a detectable decrease in at least one dimension of a bone as determined by standard radiographic measures.

The term “abnormally increased synovial cell density,” as used herein, refers to a condition in which the synovial tissue of a joint contains a number of synovial cells that is at least ten-times higher than the number of synovial cells found in the synovial tissue of a normal, i.e., non-diseased, joint.

“Administering” means any method by which a drug interacts with a patient so as to provide a physiological effect. Examples include, but are not limited to intravenous, intramuscular or intraperitoneal administration.

The term “antagonist of FLIP activity,” as used herein, refers to the property of increasing apoptosis by impeding FLIP's inhibition of caspase-8 cleavage. The decrease in FLIP activity can be achieved either through directly interfering with FLIP's ability to inhibit apoptosis or through decreasing cellular levels of FLIP protein, thereby decreasing the amount of FLIP able to bind FADD and inhibit caspase cleavage. Inhibition need not be 100% effective in order to be antagonistic.

The term “drug” refers to a compound of any degree of complexity that can affect a biological system, whether by known or unknown biological mechanisms, and whether or not used therapeutically. Examples of drugs include typical small molecules of research or therapeutic interest; naturally-occurring factors such as endocrine, paracrine, or autocrine factors, antibodies, or factors interacting with cell receptors of any type; intracellular factors such as elements of intracellular signaling pathways; factors isolated from other natural sources; pesticides; herbicides; and insecticides. Drugs can also include, agents used in gene therapy such as DNA and RNA. Also, antibodies, viruses, bacteria, and bioactive agents produced by bacteria and viruses (e.g., toxins) can be considered as drugs. A response to a drug can be a consequence of, for example, drug-mediated changes in the rate of transcription or degradation of one or more species of RNA, drug-mediated changes in the rate or extent of translational or post-translational processing of one or more polypeptides, drug-mediated changes in the rate or extent of degradation of one or more proteins, drug-mediated inhibition or stimulation of action or activity of one or more proteins, and so forth. In some instances, drugs can exert their effects by interacting with a protein. For certain applications, drugs can also include, for example, compositions including more than one drug or compositions including one or more drugs and one or more excipients.

“Inflammatory diseases” refers to a class of diverse diseases and disorders that are characterized by any one of the following: the triggering of an inflammatory response; an upregulation of any member of the inflammatory cascade; the downregulation of any member of the inflammatory cascade. Inflammatory diseases include diabetes, arteriosclerosis, inflammatory aortic aneurysm, restenosis, ischemia/reperfusion injury, glomerulonephritis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. The singular term “inflammatory disease” includes any one or more diseases selected from the class of inflammatory diseases, and includes any compound or complex disease state wherein a component of the disease state includes a disease selected from the class of inflammatory diseases.

The term “joint,” as used herein, comprises the synovial tissue, synovial fluid, articular cartilage, bone tissues, and their cellular and extracellular composition, and the soluble mediators they contain.

The term “methotrexate resistant patient” refers to a rheumatoid arthritis patient who does not effectively respond to methotrexate treatment or who initially responds to methotrexate and becomes refractory over time.

The term “TNF-α blockade resistant patient” refers to a rheumatoid arthritis patient who does not effective respond to TNF-α blockade or who initially responds to TNF-α blockade and becomes refractory over time.

The term “TNF-α blockade cartilage nonresponder” refers to a rheumatoid arthritis patient with low initial TNF-α activity who shows decreased synovial hyperplasia, but minimal reduction in cartilage degradation in response to TNF-α blockade.

The term “TNF-α blockade hyperplasia nonresponder” refers to a rheumatoid arthritis patient with abnormally high or resistant levels of TNF-α activity who yields improvement in cartilage degradation but little decrease in synovial hyperplasia in response to TNF-α blockade.

The term “TNF-α blockade double nonresponder” refers to a rheumatoid arthritis patient with negligible initial TNF-α activity who shows poor response in both synovial hyperplasia and cartilage degradation in response to TNF-α blockade.

The term “patient” refers to any warm-blooded animal, preferably a human. Patients having rheumatoid arthritis can include, for example, patients that have been diagnosed with rheumatoid arthritis, patients that exhibit one or more of the symptoms associated with rheumatoid arthritis, or patients that are progressing towards or are at risk of developing rheumatoid arthritis.

A “pharmaceutical composition” is a drug in a formulation that is safe and suitable for administration to a patient.

As used herein, a “therapeutically effective amount” of a drug of the present invention is intended to mean that amount of the compound which will inhibit an increase in synovial cells in a rheumatic joint or decrease the rate of cartilage degradation in a rheumatic joint, or decrease IL-6 concentration in synovial tissue or decrease the rate of bone erosion, and thereby cause the regression and palliation of the pain and inflammation associated with rheumatoid arthritis.

C. In Silico Modeling of a Rheumatoid Arthritis Joint

The present invention draws upon results obtained from an in silico model of an arthritic joint. The model provides a mathematical representation of the dynamic processes related to the biological state of a human joint afflicted with rheumatoid arthritis. The main compartments contained in the computer model represent synovial tissue and cartilage at the cartilage-pannus junction of this prototypical rheumatoid arthritis joint. The current model takes into account various biological variables related to the processes involved in cartilage metabolism, tissue inflammation, and tissue hyperplasia, including the following:
macrophage population dynamics including recruitment, activation, proliferation, apoptosis and their regulation,
T cell population dynamics including recruitment, antigen-dependent and antigen-independent activation, proliferation, apoptosis and their regulation
Fibroblast-like synoviocyte (FLS) population dynamic including influx into the tissue, proliferation, and apoptosis and their regulation
chondrocyte population dynamics including: proliferation and apoptosis
synthesis and regulation of a variety of proteins, including growth factors, cytokines, chemokines, proteolytic enzymes and matrix proteins, by the different cell type represented (macrophages, FLS, T cells, chondrocytes).
expression of adhesion molecules by endothelial cells
transport of mediators between synovial tissue and cartilage
interaction between cytokines or proteases and their natural inhibitors, antigen presentation, and
binding of therapeutic agents to cellular mediators (anti-TNF-α agents, such as etanercept and infliximab, and IL-1 R antagonists, such as anakinra).

The model also monitors synovial tissue density and the vascular volume. In addition, the mathematical model can take into account the effect of therapeutic agents such as methotrexate, steroids, non-steroidal anti-inflammatory drugs, soluble TNF-α receptor, TNF-α antibody, and interleukin-1 receptor antagonists.

In silico modeling is an approach that integrates relevant biological data—genomic, proteomic, and physiological—into a computer-based platform to reproduce a system's control principles. Given a set of initial conditions representing a defined disease state, these computer-based models can simulate the system's future biological behavior, a process termed biosimulation.

1. Top-Down Approach to Modeling Rheumatoid Arthritis

The computer model of the present invention was built using a “top-down” approach that started by defining a general set of behaviors indicative of rheumatoid arthritis. These behaviors are then used as constraints on the system and a set of nested subsystems is developed to define the next level of underlying detail. For example, given a behavior such as cartilage degradation in rheumatoid arthritis, the specific mechanisms inducing that behavior are each modeled in turn, yielding a set of subsystems, which themselves are deconstructed and modeled in detail. The control and context of these subsystems is, therefore, already defined by the behaviors that characterize the dynamics of the system as a whole. The deconstruction process continues modeling more and more biology, from the top down, until there is enough detail to replicate the known biological behavior of rheumatoid arthritis.

When using a top-down approach, data is identified and collected to support two specific purposes: (1) describing basic biology and (2) describing physiological function or behavior of the whole system. Data describing physiological functions or behavior of the whole system are selected early in the development of the model. These data represent the broad range of behaviors of the models system, i.e. cartilage degradation as a measurement (behavior) of rheumatoid arthritis patients. These data are human in vivo data based on well-established clinical trials. Data describing basic biology is selected to sufficiently model the subsystems required to simulate the selected behaviors. These data can be human or animal (where human is preferred but not always available) in vivo, in vitro, or ex vivo data that provide an understanding of the underlying biology.

Through this approach, researchers can discover inconsistencies with commonly accepted, but yet unproven, hypotheses and identify key knowledge gaps from the tremendous amount of in vitro and in vivo data available to the scientist. When inconsistencies and knowledge gaps are identified, the model can be used to direct specific data collection efforts that are better focused, better designed, and the data they yield more predictive and efficiently utilized.

The top-down approach was used to develop a model of rheumatoid arthritis in a human joint. A similar model is described in co-pending U.S. patent application Ser. No. 10/154,123, published 24 Apr. 2003 as 2003-0078759. Two key clinical outcomes are of particular interest in the present model: synovial cell density and the rate of cartilage degradation.

2. Sensitivity Analysis

The explicit representation of the underlying biology of the disease allows the modulation of each subsystem alone or in combination to identify the one(s) with most impact on a specific clinical outcome, such as cartilage degradation or synovial cell density. By focusing modeling and data collection efforts on those subsystems with the greatest impact on the phenotypic onset and progression or rheumatoid arthritis, this approach can help more clearly represent the system's complexity and identify causal factors underlying the pathophysiology of rheumatoid arthritis. By modulating, in silico, each subsystem (e.g. knocking-out one cell type or intercellular mediator, or blocking one particular biological process), its contribution to the overall disease pathophysiology can be evaluated to better understand the biological phenomena driving rheumatoid arthritis, thus identifying the best and most relevant targets.

In the case of rheumatoid arthritis, the disease state can be represented as outputs associated with, for example, enzyme activities, product formation dynamics, and cellular functions that can indicate one or more biological processes that cause, affect, or are modified by the disease state. Typically, the outputs of the computer model include a set of values that represent levels or activities of biological constituents or any other behavior of the disease state. Based on these outputs, one or more biological processes can be designated as critical biological processes.

The computer model can be executed to represent a modification to one or more biological processes. In particular, a modification to a biological process can be represented in the computer model to identify the degree of connection (e.g., the degree of correlation) between the biological process and rheumatoid arthritis. For example, a modification to a biological process can be represented in the computer model to identify the degree to which the biological process causes, affects, or is modified by rheumatoid arthritis. A biological process can be identified as causing rheumatoid arthritis if a modification to this biological process is observed to produce symptoms associated with rheumatoid arthritis, i.e., increased synovial cell density, cartilage degradation and IL-6 levels in the synovial tissue. In some instances, a modification to a biological process can be represented in the computer model to identify the degree of connection between other biological processes and rheumatoid arthritis.

In some instances, identifying the set of biological processes can include sensitivity analysis. Sensitivity analysis can involve prioritization of biological processes that are associated with the disease state. Sensitivity analysis can be performed with different configurations of the computer model to determine the robustness of the prioritization. In some instances, sensitivity analysis can involve a rank ordering of biological processes based on their degree of connection to the disease state. Sensitivity analysis allows a user to determine the importance of a biological process in the context of the disease state. An example of a biological process of greater importance is a biological process that increases the severity of the disease state. Thus, inhibiting this biological process can decrease the severity of the disease state. The importance of a biological process can depend not only on the existence of a connection between that biological process and the disease state but also on the extent to which that biological process has to be modified to achieve a change in the severity of the disease state. In a rank ordering, a biological process that plays a more important role in the disease state typically gets a higher rank. The rank ordering can also be done in a reverse manner, such that a biological process that plays a more important role in the disease state gets a lower rank. Typically, the set of biological processes include biological processes that are identified as playing a more important role in the disease state.

During the process of sensitivity analysis of rheumatoid arthritis the activity of biological processes such as but not limited to monocyte recruitment, T-cell recruitment, cell apoptosis, and cytokine production are modulated (increased and decreased) in a computer model one a time. Biosimulation is then conducted and the consequence of the modulation of a single biological process at different level of stimulation or inhibition is assessed by measuring clinical outcomes such as, but not restricted to, cartilage degradation, and synovial cell density. The outcome of this analysis identified the biological process that has significant impact on the clinical outcomes.

In the present invention, sensitivity analysis identified three areas of the biology of rheumatoid arthritis having a significant impact on the disease pathophysiology: (1) macrophage apoptosis, (2) T-cell apoptosis, and (3) T-cell IL-2 production.

3. Target Identification

Based on the effects of FLIP activity inhibition as predicted by the model described above, FLIP blockade is predicted to be an effective therapy for rheumatoid arthritis.

The effects of FLIP on macrophage apoptosis, T-cell apoptosis, and IL-2 production by T-cells were quantified and explicitly represented in the computer model of rheumatoid arthritis. As the contribution of FLIP activity on each of these biological processes is not clearly characterized, a range of effects was defined in order to characterize the contribution of FLIP activity (Table 1). The “lower max effect” value represents the lowest effect documented taking in consideration possible redundancies with other proteins, the “upper max effect” is the maximal possible effect of FLIP activity on each biological process and the “most likely max effect” is the estimation of the realistic contribution of FLIP activity in each biological process, taking in consideration the in vivo environment and redundancies.

TABLE 1
Effect of FLIP Activity on Joint Model

| | Lower | Most likely | Upper |
|---|---|---|---|
| Hypothesis | max effect | max effect | max effect |
| macrophage apoptosis | 0.8x | 0.5x | 0x |
| (max intracellular protection) | | | |
| T cell apoptosis | 1.4x | 2x | 5x |
| IL-2 production | 1x | 0.3x | 0.2x |

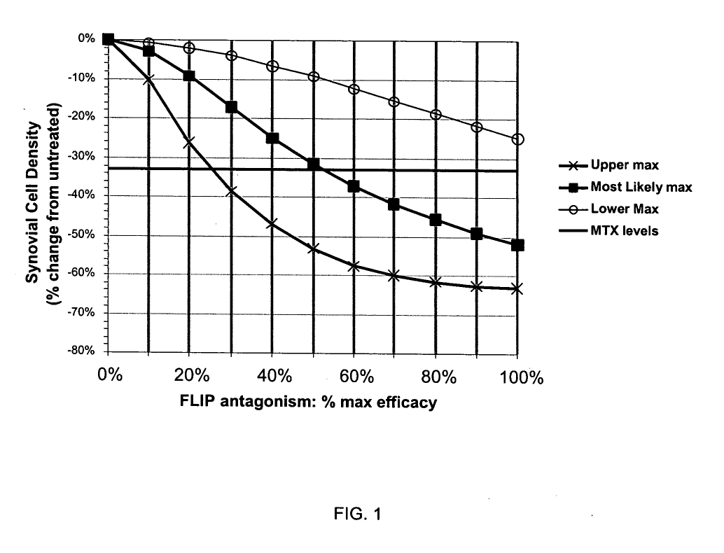

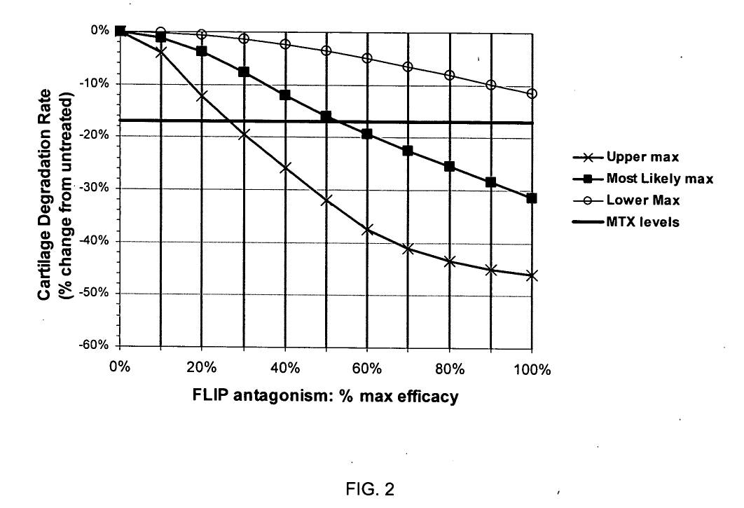

Simulation of the effect of FLIP activity on rheumatoid arthritis was then conducted by blocking FLIP in all relevant biological processes at once or in one biological process at a time or in several biological processes in combination. The results of the simulation showed that blocking FLIP activity for 6 months can improve the rheumatoid arthritis clinical outcome by reducing cartilage degradation by 12 to 46% and synovial cell hyperplasia by 24 to 63%. FIG. 1 demonstrates the effect of FLIP blockade on synovial cell density. FIG. 2 demonstrates the effect of FLIP blockade on cartilage degradation.

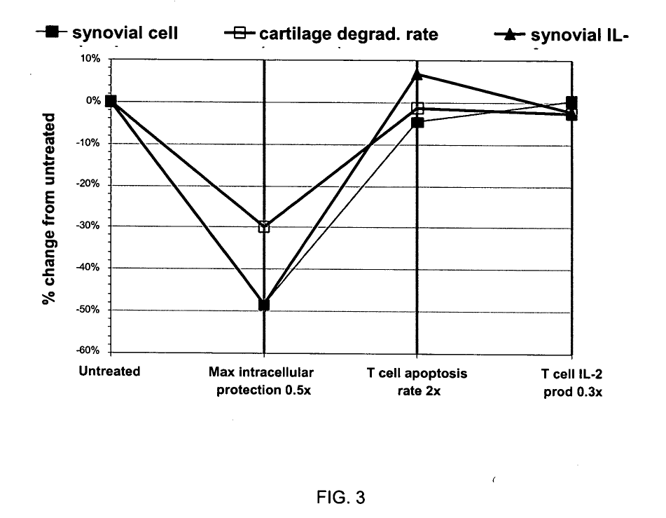

The simulation of FLIP blockade in each of the relevant biological processes, one at a time, demonstrated that macrophage apoptosis (max intracellular protection) is the main biological process driving the impact of FLIP blockade on the clinical outcome in the reference patient. The max intracellular protection is directly proportional to FLIP activity and represents the anti-apoptotic effect of FLIP that protects the macrophages against death receptor mediated apoptosis. A decrease in max intracellular protection (i.e. inhibition of FLIP activity) results in an increase in macrophage apoptosis. FIG. 3 provides the relative contribution of macrophage apoptosis (max cellular protection), T-cell apoptosis and T-cell production of IL-2 on the global effect of FLIP blockade.

Methotrexate is a common treatment for rheumatoid arthritis. Methotrexate treatment is known to decrease synovial cell density by approximately 33% and the rate of cartilage degradation by approximately 17%. At 100% efficacy, the computer model predicts FLIP antagonism will induce a greater improvement than methotrexate. A decrease in synovial cell density >33% (MTX level) can be reached in two of the three hypothesized levels: for the upper max hypothesis, efficacy of FLIP blockade has to be >25% the assessed maximum; for the most likely hypothesis, efficacy of FLIP blockade has to be >50% of maximum. Thus, if the effects of FLIP can be efficiently blocked using an antagonist of FLIP activity, the clinical outcome of this therapy in term of synovial cell density should be equal or better than that of MTX therapy.

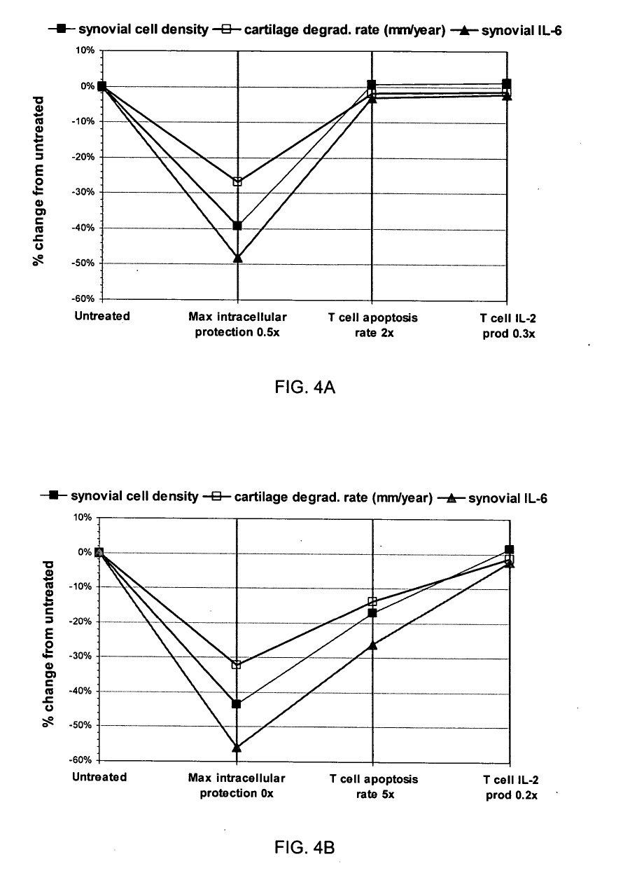

Some rheumatoid arthritis patients do not effectively respond to methotrexate treatment (initial non-responders), while other patients who initially responded to methotrexate become refractory over time (gradual non-responders). Simulation of blocking FLIP activity in a methotrexate resistant patient reveals a slightly different pattern of response than in a non-resistant patient. While similar to the standard patient, in methotrexate resistant patients, T-cell apoptosis plays a minor role in the global FLIP effect when using the upper maximum likely effect level. FIG. 4A illustrates the relative contribution of macrophage apoptosis (max cellular protection), T-cell apoptosis and T-cell production of IL-2 on the global effect in a methotrexate resistant patient utilizing the most likely maximum effect of FLIP blockade. FIG. 4B illustrates the relative contribution of macrophage apoptosis (max cellular protection), T-cell apoptosis and T-cell production of IL-2 on the global effect in a methotrexate resistant patient utilizing the upper maximum effect of FLIP blockade.

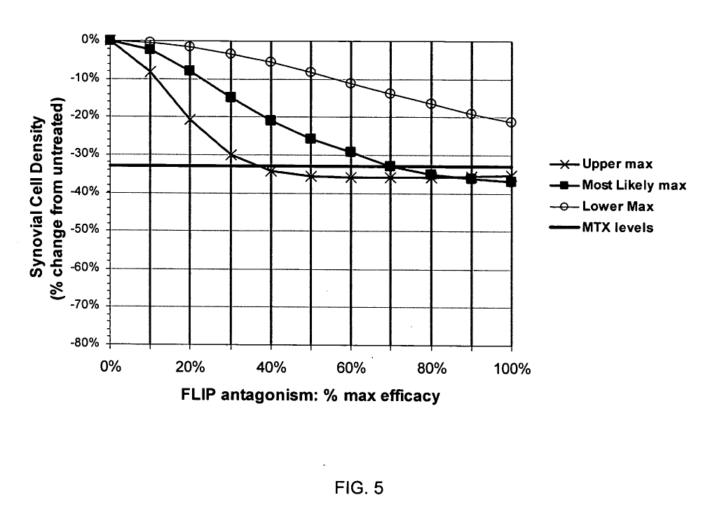

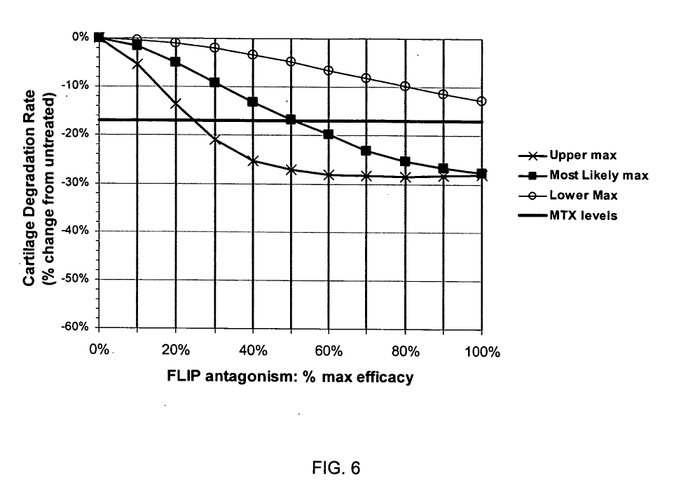

The results of the simulation showed that blocking FLIP activity for 6 months in a methotrexate resistant patient could improve the rheumatoid arthritis clinical outcome by reducing cartilage degradation by 13 to 28%, and synovial cell hyperplasia by 21 to 36%. FIG. 5 demonstrates the effect of FLIP blockade on synovial cell density in a methotrexate resistant patient. FIG. 6 demonstrates the effect of FLIP blockade on cartilage degradation in a methotrexate resistant patient.

Application of the in silico model of rheumatoid arthritis provided evidence that antagonism of FLIP activity is a promising therapeutic strategy for patients suffering from rheumatoid arthritis.

4. Thresholds

Given the model's sensitivity to macrophage apoptosis-and a desire not to overestimate potential effects of FLIP blockade-the most likely maximum effect on macrophage apoptotic signaling includes a level of redundancy from downstream regulators that blocks ˜50% of the death-receptor signaling that would otherwise occur in the model if FLIP effects were completely blocked. The value of 0.5× was selected to be somewhat conservative in estimating potential benefits. There is some indirect experimental evidence for the rationale of this level of redundancy: Zhang et al. (J. Immunol. 166:4981-4986 (2001)) observed ˜0.7× change in apoptosis rate in response to Fas signaling when they over-expressed BAR, a downstream apoptotic modulator, in 293T cells. This corresponds to the computed change in macrophage apoptosis when death-receptor signaling is modulated by 0.5×, because of the unmodulated NO-mediated apoptotic effects and feedbacks through reduced TNFα and IL-1 autocrine apoptotic signaling.

While redundant regulation of the death receptor signaling pathway seems likely, there is little quantitative direct evidence for it. Thus, the assumed upper maximum effect of FLIP blockade corresponds to completely unmodulated death-receptor signaling in the model. When assuming that FLIP is completely non-redundant in the model of a rheumatoid joint, complete inhibition of FLIP resulted in approximately a 9-fold increase in macrophage apoptosis in the joint model. There is some experimental evidence for the possibility of this level of effect. Perlman et al. (Arthritis Rheum. 44:21-30 (2001)) reported about the same change when they stimulated RA patients' synovial macrophages with a Fas agonist with and without bisinodylmaleimide I (BIS), which blocked FLIP expression. Because of the non-specific effect of BIS, a 9-fold effect is the realistic an upper maximum. The lower maximum assumes that redundant mechanisms can compensate for 80% of the effect of FLIP blockade. Table 2 provides the percentage of FLIP inhibition necessary to achieve significant clinical improvement.

TABLE 2
Percentage inhibition in FLIP activity to
achieve significant clinical improvement

| | Upper Max | | Lower |
|---|---|---|---|
| | Effect: | Most likely | Max Effect: |
| Hypothesis | No redundancy | Effect | High redundancy |
| Synovial cell density | 25% | 50% | ≧100% |
| Cartilage degradation | 25% | 50% | ≧100% |

Thus a compound useful in the treatment of rheumatoid arthritis can be identified by the property of decreasing FLIP activity by at least 25%. More preferably, useful compounds identified by the methods of the invention will decrease FLIP activity by at least 50%. Most preferably, the useful compounds identified by the methods of the invention will decrease FLIP activity by at least 95%.

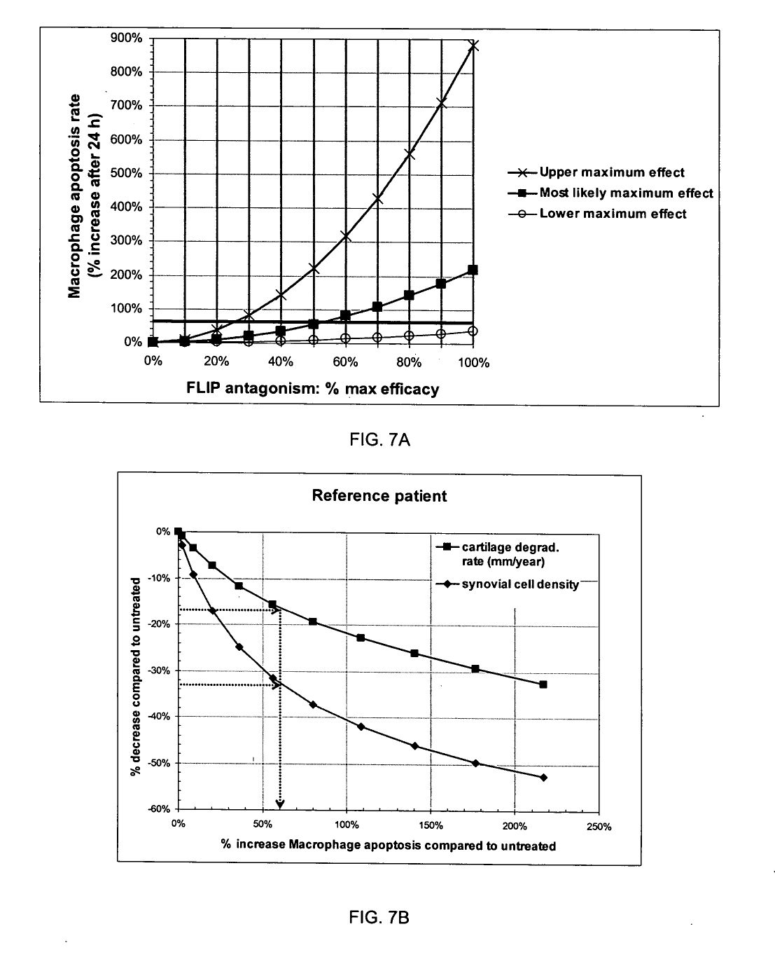

Although the amount of FLIP inhibition correlates with increased apoptosis, the increases in apoptosis are not linearly related to FLIP inhibition. FIG. 7A provides a comparison of FLIP inhibition with expected increase in macrophage apoptosis. FIG. 7B provides a comparison of macrophage apoptosis levels with the therapeutic indices of synovial cell density and cartilage degradation. The model found that to achieve a significant improvement after 6 months FLIP antagonism in synovial cell density (>33%, level measured after 6 months methotrexate therapy) and in cartilage degradation (>17%, level measured after 6 months methotrexate therapy) in the reference patient, macrophage apoptosis must increase by approximately 60% after 24 hours of FLIP inhibition. Thus a compound useful in the treatment of rheumatoid arthritis can be identified by the property of increasing macrophage apoptosis by at least 50%. More preferably useful compounds identified by the methods of the invention will increase macrophage apoptosis by at least 100%. Most preferably, the useful compounds identified by the methods of the invention will increase macrophage apoptosis by at least 200%.

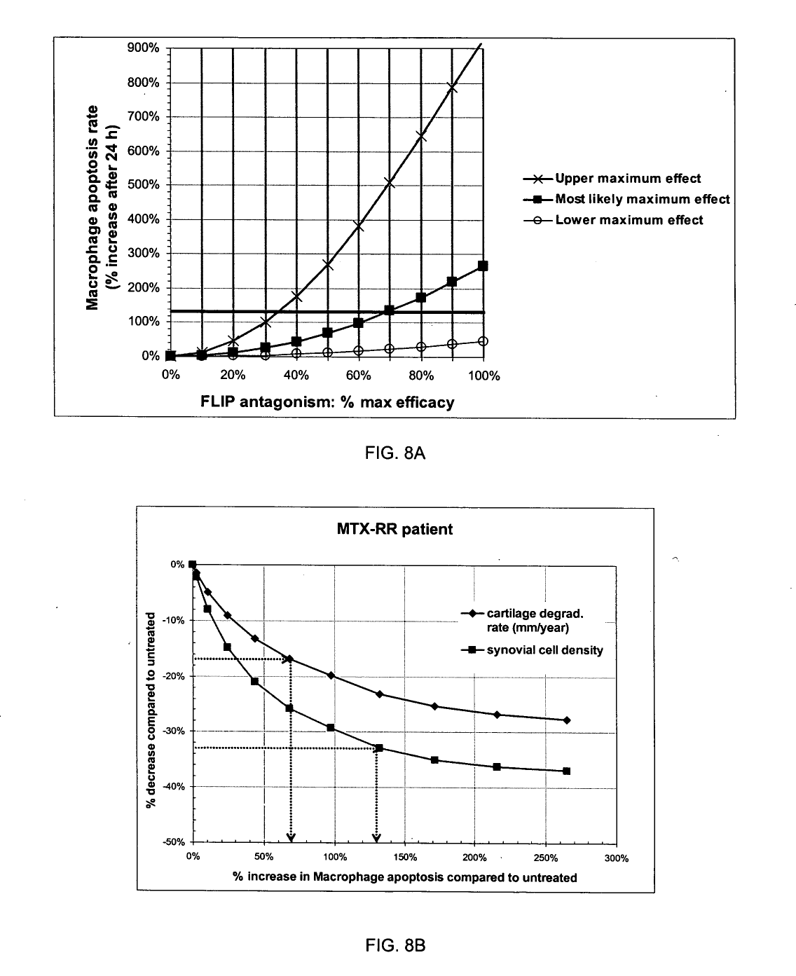

A similar analysis for a methotrexate resistant patient provides slightly different thresholds for FLIP inhibition. Table 3 provides the percentage of FLIP inhibition necessary to achieve significant clinical improvement in a methotrexate resistant patient. Consistent with the findings illustrated in FIG. 8B (cf. FIG. 7B), the synovial cell density in patients who are resistant to methotrexate is less responsive to FLIP antagonism than that of a methotrexate-responsive patient.

TABLE 3
Percentage inhibition in FLIP activity to achieve significant clinical
improvement for a methotrexate resistant patient

| | Upper | | Lower |
|---|---|---|---|
| | Max Effect: | Most likely | Max Effect: |
| Hypothesis | No redundancy | Effect | High redundancy |
| Synovial cell density | 30% | 65% | ≧100% |
| Cartilage degradation | 25% | 50% | ≧100% |

The model shows that to achieve a significant improvement in cartilage degradation in a methotrexate resistant patient, macrophage apoptosis must increase by approximately 70% after 24 hours of FLIP inhibition. However, to achieve a significant improvement in synovial cell density (>33%), macrophage apoptosis must increase by approximately 130%. FIG. 8A provides a comparison of FLIP inhibition with the expected increase in macrophage apoptosis.

D. FLIP

Apoptosis is a physiologic process that mediates the programmed death of cells. It is a highly selective way of eliminating aged and injured cells, thus controlling the regeneration of tissue. Apoptotic cells show characteristic morphologic and molecular features that include cell shrinkage accompanied by transient but violent bubbling and blebbing from the surface, condensation of chromatin, DNA fragmentation, alterations in the composition of the cell membrane, and ultimately separation of the cell into a cluster of membrane-bound bodies. The apoptotic bodies undergo phagocytosis by macrophages, which recognize apoptotic cells through specific changes in the composition of their outer cell membranes, e.g., increased levels of phosphatidylserine.

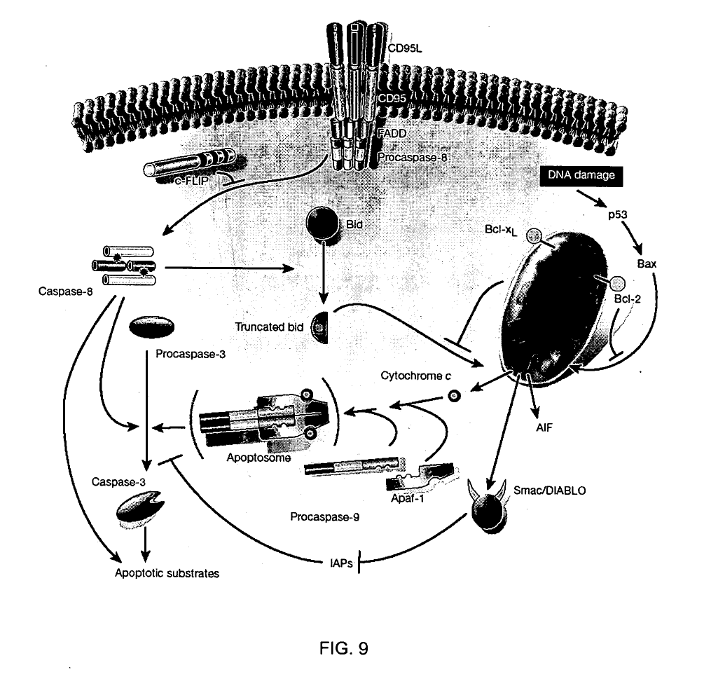

Apoptosis can be induced by internal mitochondrial-dependent and external death receptor-dependent pathways (FIG. 9). These pathways are distinct in terms of initiation, but ultimately trigger the caspase cascade leading to the classic symptoms of apoptosis.

The external pathway is initiated by stimulation of a cell membrane-associated death receptor (DR). To date, many DRs have been described. The best characterized are TNF-R1, TRAIL-R1/R2 and Fas (apoptosis antigen-1 [APO-1] or CD95), which have been shown to play a crucial role in both immune cell-mediated cytotoxicity and down regulation of immune responses. Upon binding with their cognate ligands, the DRs form aggregates that enable the recruitment of the adapter molecule FADD (Fas-associated death domain) and the initiator protease caspase-8 (also known as FLICE [FADD-like IL-1β-converting enzyme]). Cleavage of caspase-8 initiates the caspase cascade, culminating in the cleavage of various substrates, such as lamins, fodrin, gelsolin, actin and the inhibitor of caspase-activated DNase (ICAD), leading to cell dissolution and death. Among the molecules regulating DR-mediated apoptosis, FLIP appears to play a central inhibitory role by blocking the early events of the DR signaling cascade. FLIP inhibits the activation of caspase-8, one of the earliest signaling events in the DR mediated apoptotic pathway.

The FLICE-inhibitory proteins were first identified as a new class of viral anti-apoptotic protein (v-FLIP). A cellular homolog, c-FLIP, was recently identified by several groups as one of the main physiologic inhibitors of DR mediated apoptosis. Other names for FLIP include Casper (caspase-8-related protein), CLARP (caspase-like apoptosis-regulatory protein), FLAME-1 (FADD-like anti-apoptotic molecule 1), I-FLICE (inhibitor of FLICE), CASH (caspase homolog), MRIT (MACH (MORT-associated CED-3 homolog)-related inducer of toxicity) and Usurpin.

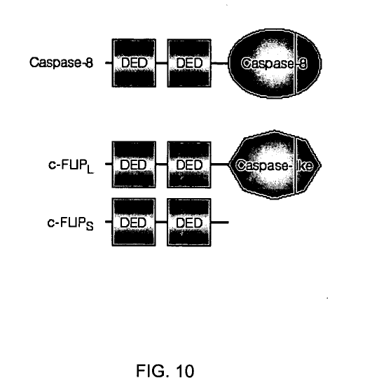

FLIP is an intracellular protein with structural homology with the apoptosis-initiators caspase-8 and casepase-10 (FIG. 10). Two splice variants of FLIP are expressed in vivo: short FLIP (FLIPS) of 26 kDa and long FLIP (FLIP_{L}) of 55 kDa. Both FLIP_{S} and FLIP_{L} inhibit apoptosis induced by Fas, TRAIL-R1, TRAIL-R2, TRAMP and TNF-R1. However, FLIP_{L} is considerably more potent than FLIP_{S}. FLIP contains two serial N-terminal death effector domains (DEDs) followed by a C-terminal extension comprising a caspase-homologous domain similar to caspase-8 and caspase-10. However, owing to the substitution of several amino acids conserved in caspases, FLIP has no proteolytic activity. Via its DED, both FLIP isoforms bind to the Fas-associated-death domain (FADD), an adapter protein that mediates death receptor signaling from Fas and TNF receptors, among others. FLIP isoforms typically interfere with the autoproteolytic activation of pro-caspase-8 and pro-caspase-10, thus inhibiting the consequent apoptotic signaling cascade.

FLIP is a short-lived protein, the expression of which can be inhibited by a variety of substances, e.g., oxidized low-density lipoproteins, chemotherapeutic agents including doxorubicin, 5-FU, and cisplatin, p53, synthetic peroxisome proliferated-activated receptor (PPAR) ligands, sodium butyrate, IFN-β, E1A, and hemin. FLIP also is believed to be post-translationally regulated by phosphorylation.

FLIP also is reported to up-regulate NFκB expression (Kataoka et al., Curr Biol. 10:640-8. (2000), Hu et al., J Biol Chem. 275:10838-44 (2000)), and to be upregulated by NFκB (Micheau et al., Mol Cell Biol. 21:5299-305 (2001), Kreuz et al., Mol Cell Biol. 21(12):3964-73 (2001)). These effects apparently account, respectively, for the proliferative effects of FLIP in lymphocytes, and possibly a significant portion of the anti-apoptotic effects of NFκB. FLIP also up-regulates ERK expression (Kataoka et al., 2000; Micheau et al., 2001).

It has been suggested that deficient apoptosis plays a role in rheumatoid arthritis. For example, it has recently been demonstrated that arthritic lesions can be induced by persistent engrafted syngeneic lymphocytes overexpressing FasL (Bonardelle, et al., J. Rheumatol. 28:956-961 (2001)). TRAIL, a ligand for cell-surface death receptors, has been shown to play an important role in the clearance of autoreactive synovium-infiltrating cells in rheumatoid arthritis (Lamhamedi-Cherradi, et al., Nat. Imunol. 4:255-260 (2003)). Finally, FLIP expression is increased in synovial biopsy specimens from patients with rheumatoid arthritis, especially in synovial macrophage cells (Perlman, et al., Arthritis Rheum. 44:21-30 (2001)). However, even though FLIP expression has been correlated with rheumatoid arthritis, inhibition of FLIP has not been shown to alleviate the symptoms of the disease. Further, the literature fails to provide any guidance regarding to what extent FLIP must be inhibited in order to alleviate symptoms of rheumatoid arthritis.

E. Methods of Identifying FLIP Antagonists and Anti-Rheumatic Drugs

As described above, inhibiting macrophage apoptosis is the major contributor to the benefits of FLIP blockade. While not limited to any theory, it is believed that FLIP acts by binding to FADD, thus physically blocking pro-caspase-8 binding to FADD. Caspase-8 binding to FADD enhances auto-cleavage of caspase-8 and thus initiation of the caspase cascade leading to apoptosis. FLIP activity can be assayed by any of the apoptosis assays that are well known in the art. See, e.g., Steensma, et al., Methods Mol Med. 85:323-32 (2003) or Willingham, J. Histochem. Cytochem. 47:1101-1109 (1999). FLIP activity can be decreased by either by interfering with FLIP binding to FADD or by merely decreasing the level of FLIP expression in the target cell.

Thus one aspect of the invention provides methods for screening a collection of compounds for a compound useful in the treatment of rheumatoid arthritis comprising, (a) comparing an amount of FLIP activity in the presence of the compound with an amount FLIP activity in the absence of the compound; and (b) selecting the compound as useful in the treatment of rheumatoid arthritis when the amount of FLIP activity in presence the of the compound is at least 25% lower than the amount of FLIP activity in the absence of the compound.

FLIP activity can be measured directly by determining the ability of FLIP to bind to FADD. The amount of binding can be determined by any of a variety of methods well know in the art, including co-immunoprecipitation of FLIP and FADD or BIACORE measurements of the interaction between FLIPP and FADD. Alternatively, FLIP activity can be measured by determining the level of caspase-8 activity. Because caspase-8 is a protease, its activity is susceptible to a number of colorimetric and spectrophotometeric detection methods. The primary effect of FLIP activity is to inhibit death receptor mediated apoptosis. Therefore, inhibition of FLIP can also be determined by detecting an increase in apoptosis.

Therefore, one aspect of the invention provides methods of screening a collection of compounds for a compound useful in the treatment of rheumatoid arthritis comprising comparing an amount of macrophage apoptosis in the presence of the compound with an amount of macrophage apoptosis in the absence of the compound, and selecting the compound as useful in the treatment of rheumatoid arthritis when the amount of macrophage apoptosis in the presence of the compound is at least 50% greater than the amount of macrophage apoptosis in the absence of the compound.

1. FLIP Expression

Methods of determining expression levels of proteins are well known in the art. The method described herein it just one of the many acceptable methods for determining FLIP expression levels.

Monocytes or macrophages can be isolated from synovial fluid or peripheral blood mononuclear cells from RA patients or healthy donors by either Percoll or Histopaque (Sigma Chemical Co.) gradient centrifugation or countercurrent centrifugal elutriation (Beckman-Coulter). Monocytes can be differentiated in macrophages with RPMI containing 20% heat-inactivated fetal bovine serum (FBS) plus 1 μg/ml polymyxin B sulfate (Sigma Chemical Co.) in 24-well plates (Costar). The macrophages are incubated with a compound of the invention for periods of time ranging from one hour to several days. After incubation, the cells are lysed by any suitable method to produce a cell lysate. The amount FLIP expression can be determined via Western Blot, immunoprecipitation or any other quantitative procedure utilizing anti-FLIP antibodies. Suitable anti-FLIP antibodies include Dave-2 or clone NF6 (Axorra LLC, San Diego, Calif.). Any antibody or antibody fragment, polyclonal or monoclonal antibody specific for FLIP may be used to quantify FLIP expression. Appropriate negative controls, including cells treated identically to the test cells with the exception of exposure to the test compound should be performed in order to identify alterations in FLIP expression due to exposure to the compound rather than manipulations of the cells during experimentation.

Various procedures, well known in the art, may be used for the production of polyclonal antibodies to FLIP. For example, for the production of polyclonal antibodies, various host animals, including but not limited to rabbits, mice, rats, etc., can be immunized by injection with FLIP or a derivative thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

A monoclonal antibody (mAb) to FLIP can be prepared by using any technique known in the art, which provides for the production of antibody molecules by continuous cell lines in culture. These include but are not limited to, the hybridoma technique originally described by Kohler and Milstein (Nature 256:495-497 (1979)), the more recent human B cell hybridoma technique (Kozbor et al., Immunology Today 4:72 (1983)), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA and, IgD and any subclass thereof. The hybridoma producing the mAbs of use in this invention may be cultivated in vitro or in vivo.

2. Caspase-8 Activity

FLIP has been identified as an inhibitor of caspase-8, also known as FLICE. Therefore measurement of an increase in caspase-8 activity is a preferred surrogate for measuring decreases in FLIP activity. Caspase-8 specifically cleaves a peptide with the sequence IETD (Ile-Glu-Thr-Asp, SEQ ID NO: 1). Cleavage by caspase-8 can be detected utilizing colorimetric or fluorescent methods well known in the art.

In an exemplary method for detecting caspase-8 activity, macrophages can be isolated from synovial tissue of rheumatoid or healthy patients or can obtained by differentiation of peripheral blood monocytes. The cells are lysed utilizing any appropriate method, such as NP-40 lysis. The test compound can be incubated with the macrophages for a period of one to 24 hours prior to preparation of the cell lysate. Optionally, the incubation may also include a death receptor-dependent inducer of apoptosis such as Fas ligand, TRAIL, TNF-α or an anti-death receptor (e.g., TNF-R1, Fas, TRAIL-R or DR6) antibody. A caspase-8 substrate, such as the synthetic peptide, IETD (SEQ ID NO: 1), conjugated to a detectable marker is added to the cell lysate. The peptide substrate is conjugated to the detectable marker in such a fashion that when the peptide substrate is cleaved, the detectable marker becomes detectable or alters a detectable property so that the amount of cleavage can be quantified. Examples of suitable substrates include IETD-pNA (p-nitroanilide) and IETD-AMC (7-amino-4-methylcoumarin). Free pNA is detectable at 405 nm. Free AMC is detectable with a 380 nm excitation filter and 460 nm emission filter. Commercial kits for the detection of caspase-8 activity are available, e.g., from Clontech (ApoAlert Caspase-8 Colorimetric Assay Kit or ApoAlert Caspase Assay Plates).

3. DNA Fragmentation Assays

Loss of DNA integrity is another characteristic of apoptosis. When DNA extracted from apoptotic cells is analyzed using gel electrophoresis, a characteristic “ladder” of DNA fragments is seen. However, extraction of DNA from cells is a time consuming process and alternative methods are equally suitable for detecting the characteristic fragmentation of DNA in apoptotic cells. DNA fragmentation can be detected by a variety of assay including propidium iodide assays, acridine orange/ethidium bromide double staining, the TUNEL and ISNT techniques, and the assays of DNA sensitivity to denaturation.

4. Annexin V Assays

Externalization of phosphatidylserine (PS) and phosphatidylethanolamine is a hallmark of the changes in the cell surface during apoptosis. Annexin V is a 35-36 kDa Ca²⁺-dependent, phospholipid binding protein that has a high affinity for PS and binds to cells with exposed PS. Annexin V may be conjugated to any of a variety of markers to permit it to be detected by microscopy or flow cytometry. For use in methods of identifying compounds that inhibit FLIP activity or methods of screening for compounds that inhibit FLIP activity, it is preferable to use fluorescently labeled annexin V detected by flow cytometry.

Macrophages are obtained as discussed above from either rheumatoid or healthy subjects. Cells are incubated with the test compound for one to 24 hours, optionally in the presence of a DR-dependent inducer of apoptosis. The number of cells committed to apoptosis is determined by staining with labeled annexin V and a vital dye, such as propidium iodide (PI) or 7-amino-actinomycin D (7-AAD). Because externalization of PS occurs in the earlier stages of apoptosis, annexin V staining precedes the loss of membrane integrity that accompanies the latest stages of cell death resulting from either apoptotic or necrotic processes. Therefore, staining with annexin V in conjunction with vital dyes such as propidium iodide (PI) or 7-amino-actinomycin D (7-AAD) permits identification of early apoptotic cells (annexin V-positive and vital dye-negative).

F. Methods of Treatment

In one aspect, the invention provides methods of alleviating at least one symptom of an inflammatory disease, such as rheumatoid arthritis, comprising administering a therapeutically effective amount of an antagonist of FLIP activity to a patient having an inflammatory disease. The invention also provides methods for alleviating at least one symptom of rheumatoid arthritis comprising administering a therapeutically effective amount of an antagonist of FLIP activity to a patient having rheumatoid arthritis, wherein the antagonist decreases FLIP activity by at least 25%. Preferably, the antagonist decreases FLIP activity by at least 50%. More preferably, the antagonist decreases FLIP activity by at least 70%. Most preferably, the antagonist of FLIP activity decreases FLIP activity by at least 95%. The antagonist of FLIP activity maybe a protein, nucleic acid or small molecule inhibitor. A preferred protein antagonist is oxidized low-density lipoprotein, ectopic-p53, IFN-β, PPAR ligand, E1A, or hemin. Preferred nucleic acid antagonists include antisense inhibitors of any sequence complementary to FLIP mRNA, but preferably is 5′-GACTTCAGCAGACATCCTAC-3′ (SEQ ID NO: 2). The invention also encompasses methods of decreasing synovial cell density and methods of decreasing cartilage degradation by administering a therapeutically effective amount of an antagonist of FLIP activity, wherein the antagonist decrease FLIP activity by at least 25%, preferably at least 50%, more preferably at least 70% and most preferably at least 95%.

Antisense inhibitors have been shown to be capable of interfering with expression of target proteins. See Cohen, “Designing antisense oligonucleotides as pharmaceutical agents,” Trends Pharmacol Sci. 10:435-7(1989) and Weintraub, “Antisense RNA and DNA,” Sci Am. 262:40-6 (1990), both incorporated herein by reference. Antisense inhibitors of c-FLIP are described in detail in Siegmund, et al., Molec. Med. 8:725-732 (2002) and PCT publication WO 02/24717, incorporated by reference herein.

A compound useful in this invention is administered to a patient in a therapeutically effective dose by a medically acceptable route of administration such as orally, parenterally (e.g., intramuscularly, intravenously, subcutaneously, intraperitoneally), transdermally, rectally, by inhalation. The dosage range adopted will depend on the route of administration and on the age, weight and condition of the patient being treated.

Various delivery systems are known and can be used to administer a composition of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

G. Combination Therapies

In one aspect, the invention provides methods of alleviating at least one symptom of rheumatoid arthritis, comprising administering an antagonist of FLIP activity and an anti-inflammatory drug to a patient having rheumatoid arthritis. Preferably, the anti-inflammatory drug is selected from the group of methotrexate, an interleukin-1 receptor antagonist and a steroid. More preferably, the anti-inflammatory drug is methotrexate, Anakinra or prednisone. In one embodiment of the invention, the patient is resistant to methotrexate or to TNF-α blockade.

Various treatment protocols were simulated alone, or in combination with antagonism of FLIP activity. The effects of several therapies are represented in the model. The model reproduces the impact of treatment with (1) non-steroidal anti-inflammatory drugs (NSAIDs; e.g., indomethacin), (2) Etanercept, a soluble type II TNF-α receptor, (3) Infliximab, a monoclonal antibody to TNF-α, (4) methotrexate (MTX), (5) glucocorticoids (e.g., methylprednisolone), and (6) Anakinra, an IL-1 receptor antagonist (IL-1Ra).

Each therapy is implemented based on its mode of action, molecular activity, and pharmacokinetic properties as well as its recommended clinical dosing regimen. To determine the importance of time-dependent variation in drug exposure associated with the clinically recommended periodic drug administration, we compared simulation results based on the clinical schedule with results for a constant-concentration continuous dose with an equivalent serum area-under-the-curve (AUC) net drug exposure. Simulation results for the two different administration schedules differed only minimally. In order to simplify presentation of results by eliminating transient effects due to periodic administration, results discussed herein are based on continuous dose therapy simulations.

The impact of the simulated treatments results from the implemented molecular activity. For example, Etanercept is modeled as binding and neutralizing TNF-α; any subsequent changes in hyperplasia, cartilage degradation, or other measurements are a secondary consequence of this reduction in free, active TNF-α, rather than a direct or specified effect of Etanercept. The effects directly implemented for each therapy are as follows:

The primary, common mode of action of NSAIDs is the inhibition of the cyclo-oxygenase (COX) pathways and synthesis of their downstream products, especially prostaglandin-E2 (PGE2). The model implementation of NSAIDs is based on in vitro data on the dose-dependent inhibition by NSAIDs of PGE2 synthesis in macrophages, FLS, and chondrocytes. Simulation results presented are for a constant continuous dose with serum AUC drug exposure equivalent to that achieved with a dosing schedule of 50 mg indomethacin, administered orally 3 times a day.

Etanercept and Infliximab (exogenous sTNF-RII and anti-TNF-α antibody respectively) are modeled as binding and neutralizing soluble TNF-α. The binding of these agents to TNF-α is modeled using appropriate values for binding rate parameters of each molecule. The net binding rate of soluble receptors (or anti-TNF-α) to TNF-α is calculated as the difference between the binding and dissociation rates as follows:

$$\frac{ⅆ}{ⅆ t} [ TNF α \text{:} sTNFR ] = {k}_{on} [ TNF α ] [ sTNFR ] - {k}_{off} [ TNF α \text{:} sTNFR ] \qquad ( eq . 1 )$$

where kₒₙ=constant of association between sTNF-R and TNF-α
k_{off}=constant of dissociation between sTNF-R and TNF-α
[TNFα]=concentration of free TNF-α
[sTNFR]=concentration of free soluble TNF-R
[TNFα: sTNFR]=concentration of bound complexes

Simulation results presented are for a constant continuous dose of Etanercept with serum AUC drug exposure equivalent to that achieved with a dosing schedule of 25 mg, administered subcutaneously twice a week.

Methotrexate therapy is implemented based on in vitro data that quantify its direct effects on particular cellular functions, including dose-dependent inhibition of T cell and FLS proliferation, mediator synthesis, and apoptosis. In addition, to account for the inhibitory effect of methotrexate on vascular proliferation and vascularization, a reduction in total endothelial adhesion molecules expression is also implemented. Simulation results presented are for a constant continuous dose with serum AUC drug exposure equivalent to that of a dosing schedule of 12.5 mg/week, administered orally.

Methylprednisolone is represented by the dose-dependent modulation of various cellular mediator synthesis rates according to in vitro data. Effects on other cell functions are not directly modeled but may arise from altered mediator-dependent regulation. Simulation results presented are for a constant continuous dose with serum AUC drug exposure equivalent to that of a dosing schedule of 5 mg methylprednisolone, administered orally once a day.

Anakinra, like endogenous IL-1Ra, is modeled as reducing the impact of IL-1β on all cellular functions. This is implemented by calculating an “effective” IL-1β concentration that has been adjusted to account for the impact of reduced receptor binding in the presence of the instantaneous concentration of receptor antagonist. Simulation results presented are for a constant continuous dose with serum AUC drug exposure equivalent to that of a dosing schedule of 100 mg Anakinra, administered subcutaneously once a day.

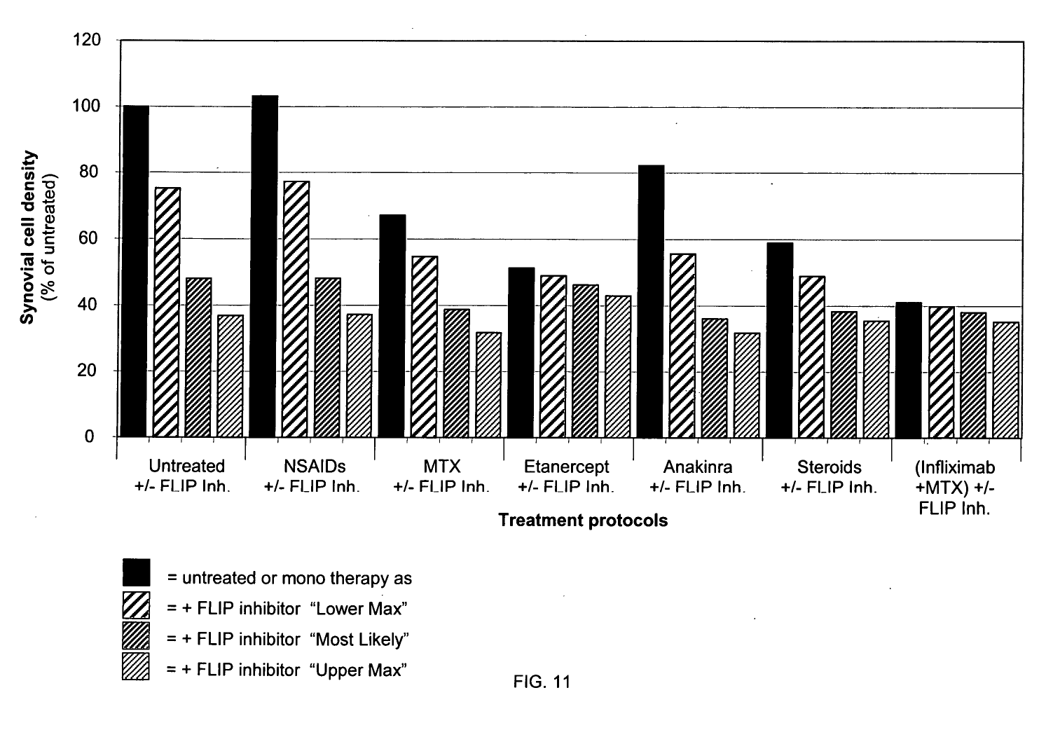

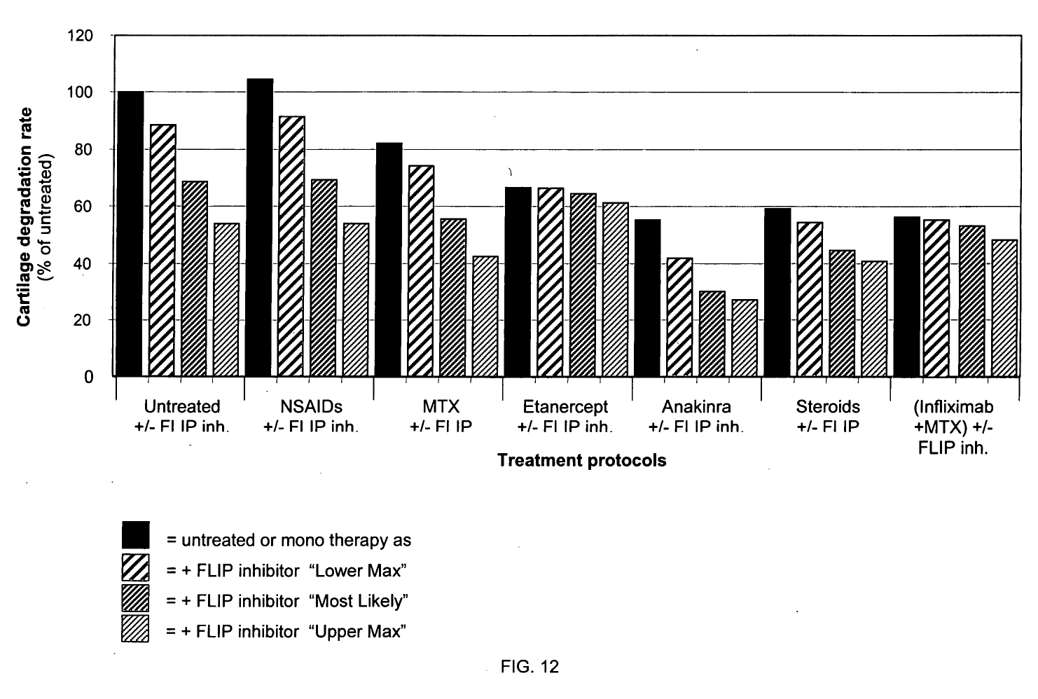

Simulation of the effect of treatment on the progression of rheumatoid disease in a virtual patient was conducted by simulating the rheumatoid arthritis in the virtual patient for one year without treatment to establish a baseline in the model. Then either no treatment, a current treatment protocol or a current protocol in combination with FLIP antagonism was modeled. FLIP antagonism was modeled assuming 100% inhibition of FLIP activity having the “most likely max effect,” which is the estimation of the realistic contribution of FLIP activity, taking in consideration the in vivo environment and redundancies. The effects of the simulated treatment (or lack of treatment) in a typical patient for six months on synovial cell density are illustrated in FIG. 11. The effects of the simulated treatment for six months on cartilage degradation are illustrated in FIG. 12. The effect of combination therapy as compared to monotherapy or treatment with FLIP antagonism alone is summarized in Table 4.

TABLE 4
Combination therapies in typical rheumatoid arthritis patient

| | Effect on synovial | | Effect on cartilage | |
|---|---|---|---|---|
| | cell hyperplasia | | degradation | |
| | | v. FLIP | v. | v. FLIP |
| Treatment | v. Tx alone | inh alone | Tx alone | inh alone |
| NSAIDs + FLIP inh | ++ | = | ++ | = |
| MTX + FLIP inh. | ++ | ++ | +/++ | ++ |
| Etanercept + FLIP inh. | =/+ | + | = | + |
| Anakinra + FLIP inh. | ++ | ++ | ++ | ++ |
| Steroids + FLIP inh. | +/++ | ++ | =/+ | +/++ |

The results of the simulation showed that blocking FLIP activity in addition to administration of an interleukin-1 receptor antagonist, such as Anakinra, can improve the rheumatoid arthritis clinical outcome by reducing cartilage degradation by 58 to 73% and synovial cell hyperplasia by 44 to 68%. Similarly, treatment with FLIP inhibition in combination with methotrexate or a steroid, such as methylprednisolone, shows decreases in synovial cell hyperplasia and cartilage degradation that cannot be achieved with a monotherapy.

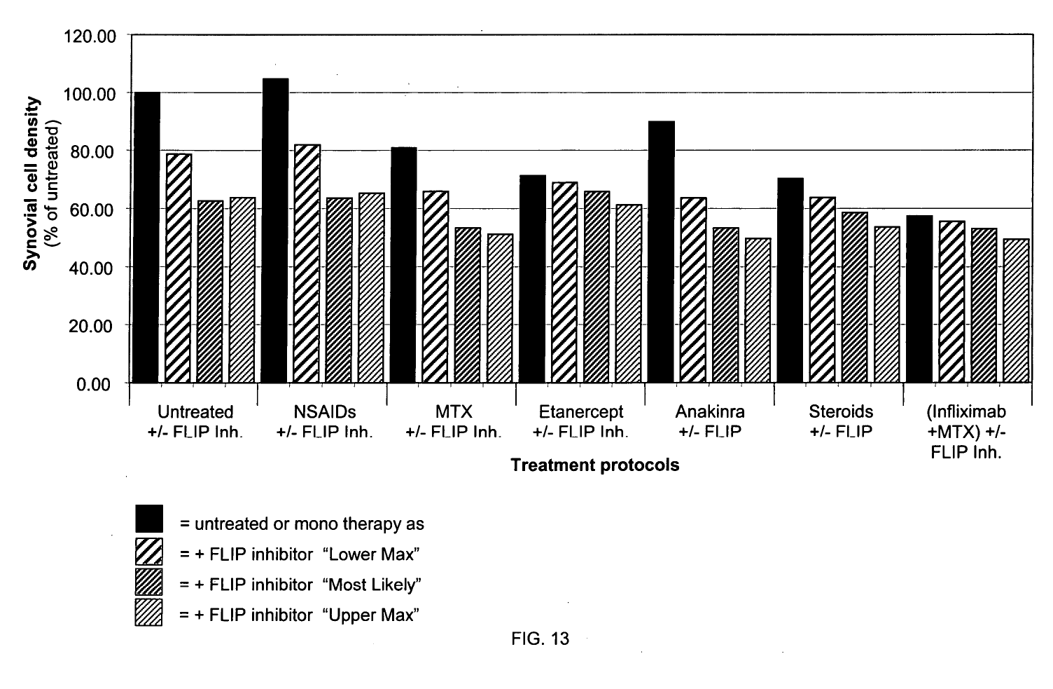

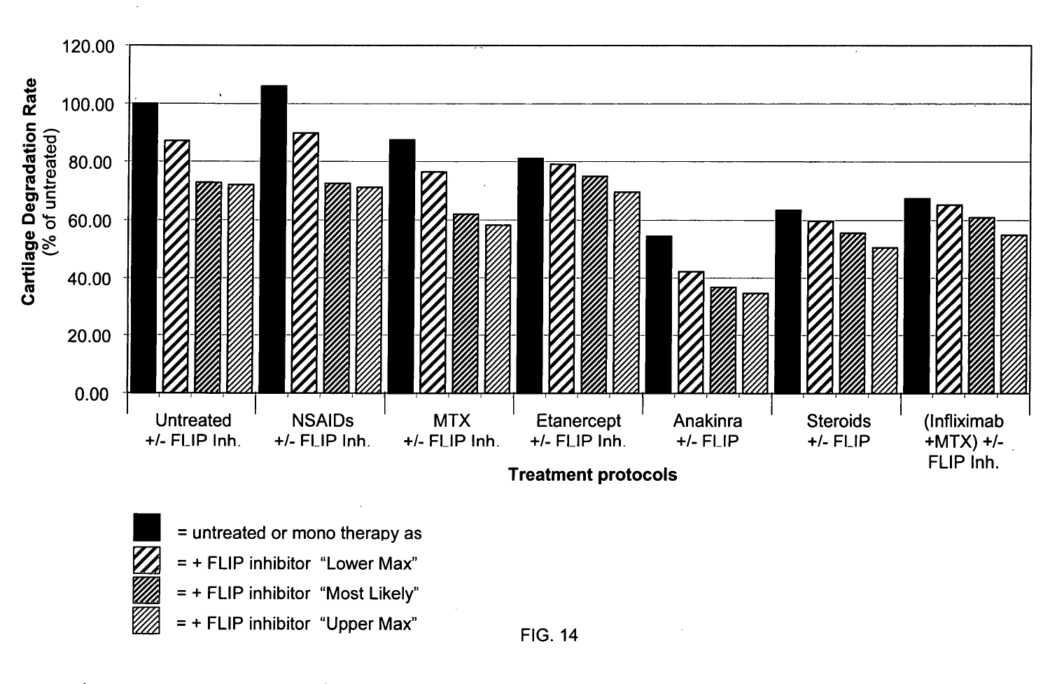

Simulation of FLIP antagonism in combination with standard anti-rheumatic treatments in a methotrexate resistant patient revealed a slightly different pattern of response than in a normal methotrexate-responsive patient. The effects of the simulated treatment (or lack of treatment) in a methotrexate resistant patient for six months on synovial cell density is illustrated in FIG. 13. The effects of the simulated treatment for six months on cartilage degradation is illustrated in FIG. 14. The effect of combination therapy as compared to monotherapy or treatment with FLIP antagonism alone in a methotrexate resistant patient is summarized in Table 5.

TABLE 5
Combination therapies in methotrexate resistant patient

| | Effect on synovial | | Effect on cartilage | |
|---|---|---|---|---|
| | cell hyperplasia | | degradation | |
| | | v. FLIP | v. | v. FLIP |
| Treatment | v. Tx alone | inh alone | Tx alone | inh alone |
| NSAIDs + FLIP inh | ++ | = | ++ | = |
| MTX + FLIP inh. | ++ | ++ | +/++ | ++ |
| Etanercept + FLIP inh. | =/+ | =/+ | =/+ | =/+ |
| Anakinra + FLIP inh. | ++ | ++ | ++ | ++ |
| Steroids + FLIP inh. | + | +/++ | =/+ | ++ |

The results of the simulation showed that blocking FLIP activity in addition to administration of an interleukin-1 receptor antagonist, such as Anakinra, can improve the rheumatoid arthritis clinical outcome by reducing cartilage degradation by 58 to 65% and synovial cell hyperplasia by 36 to 50%. Interestingly, a combination therapy comprising FLIP antagonism and administration of methotrexate to a methotrexate resistant patient can improve the rheumatoid arthritis clinical outcome by reducing cartilage degradation and synovial cell hyperplasia to a greater extent than achieved by FLIP antagonism or methotrexate treatment alone. Further the difference between a combination of FLIP antagonism and steroid treatment on clinical progression of the disease is smaller in a methotrexate resistant patient than in a normal patient (cf FIGS. 13 and 14 to FIGS. 11 and 12).

TNF-α neutralizing therapies have become increasingly important in treating rheumatoid arthritis patients. However, roughly a third of all rheumatoid arthritis patients fail to achieve a clinically significant response to TNF-α neutralizing therapies. Three potential classes of TNF-α blockade resistant patients were defined in the model described above. Synovial hyperplasia and cartilage degradation are differentially affected when TNF-α varies within different ranges, leading to the identification of three nonresponder classes within the current model. Specifically, patients with low initial TNF-α activity show decreased synovial hyperplasia, but minimal reduction in cartilage degradation in response to TNF-α blockade (cartilage nonresponders, or CNRs), while patients with negligible initial TNF-α activity show poor response in both synovial hyperplasia and cartilage degradation (double nonresponders or DNRs). Alternatively, insufficient neutralization of TNF-α in patients with abnormally high or resistant levels of TNF-α activity yields improvement in cartilage degradation but poor response in hyperplasia (hyperplasia nonresponders or HNRs). Mechanistically, in patients with low levels of TNF-α, rheumatoid disease was perpetuated by increased activity of alternate macrophage activating pathways (e.g., CD40-ligation), reduced activity of anti-inflammatory cytokines (e.g., IL-10), and increased activity of degradation-promoting cytokines (e.g., IL-1β). Nonresponding patients also showed altered responses to other therapies such as IL-1Ra (data not shown).

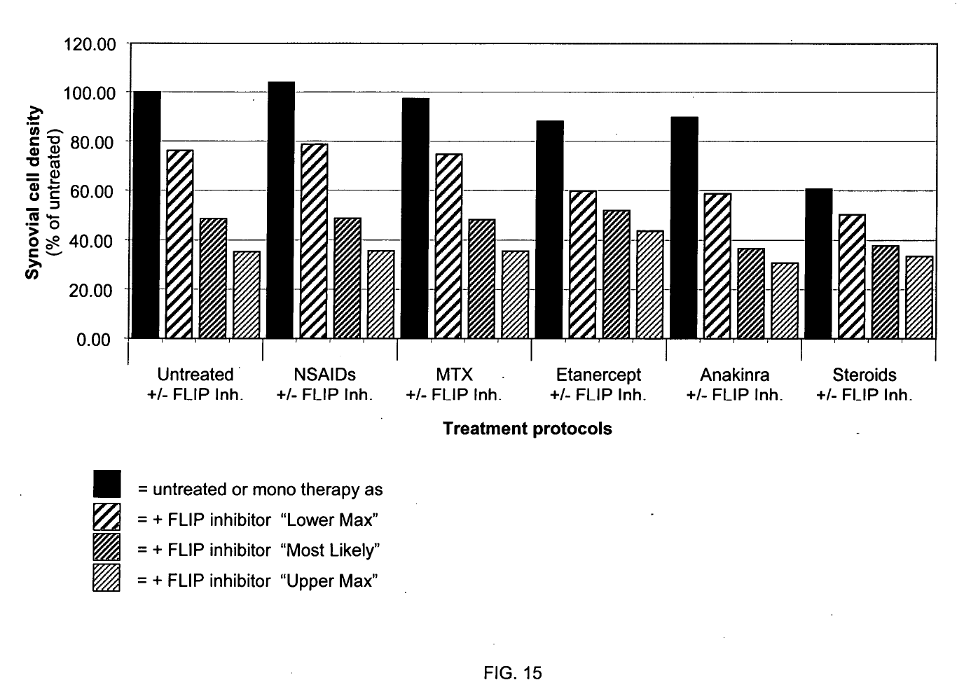

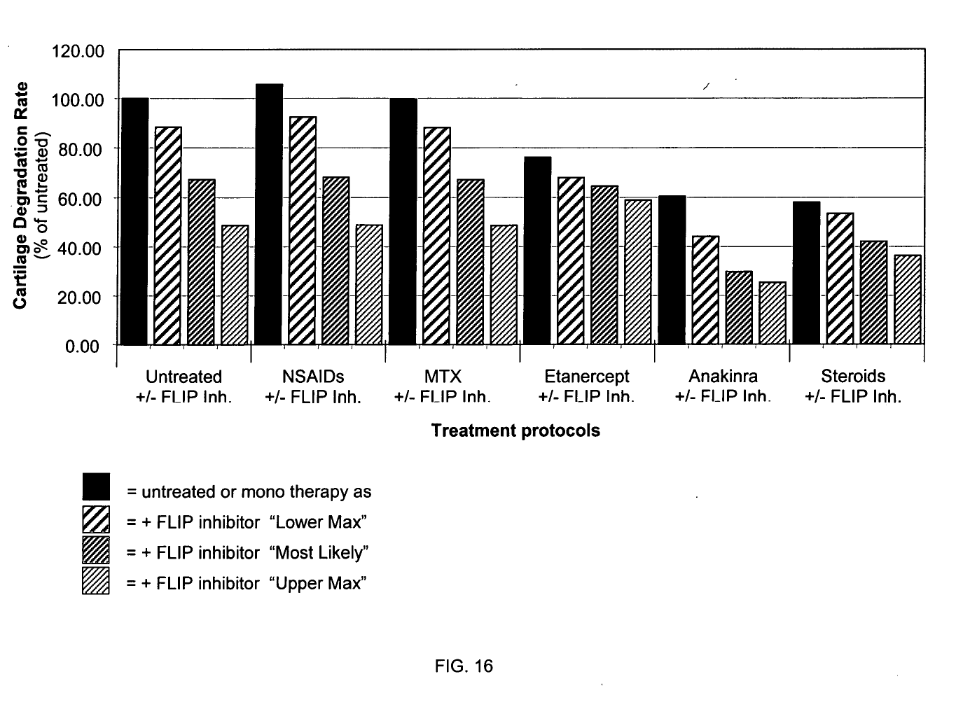

Patients who fail to achieve a significant clinical response to TNF-α blockade represent a sizable subset of the rheumatoid arthritis population. Simulation of FLIP antagonism in combination with standard anti-rheumatic treatments in a TNF-α hyperplasia nonresponder revealed a slightly different pattern of response than in a normal methotrexate-responsive patient. The effects of the simulated treatment (or lack of treatment) in a methotrexate resistant patient for six months on synovial cell density is illustrated in FIG. 15. The effects of the simulated treatment for six months on cartilage degradation is illustrated in FIG. 16. The effect of combination therapy as compared to monotherapy or treatment with FLIP antagonism alone in a methotrexate resistant patient is summarized in Table 5.

TABLE 6
Combination therapy in TNF-α blockade hyperplasia nonresponder

| | Effect on synovial | | Effect on cartilage | |
|---|---|---|---|---|
| | cell hyperplasia | | degradation | |
| | | v. FLIP | v. | v. FLIP |
| Treatment | v. Tx alone | inh alone | Tx alone | inh alone |
| NSAIDs + FLIP inh | ++ | = | +/++ | = |
| MTX + FLIP inh. | ++ | = | +/++ | = |
| Etanercept + FLIP inh. | ++ | =/+ | + | =/+ |
| Anakinra + FLIP inh. | ++ | +/++ | ++ | ++ |
| Steroids + FLIP inh. | +/++ | ++ | +/++ | ++ |

The results of the simulation showed that combination therapy comprising FLIP antagonism and administration of methotrexate to a TNF-α blockade resistant patient showed no improvement in clinical outcome as compared to FLIP antagonism alone. However, combination of FLIP antagonism with either IL-1Ra or steroid treatment can result in less synovial cell hyperplasia and lower cartilage degradation rates as compared to the monotherapy or FLIP antagonism alone. Blocking FLIP activity in addition to administration of an interleukin-1 receptor antagonist, such as Anakinra, improves the rheumatoid arthritis clinical outcome by reducing cartilage degradation by 56 to 75% and synovial cell hyperplasia by 41 to 69%.

An antagonist of FLIP activity and another disease modifying anti-rheumatoid drug are administered concurrently. “Concurrent administration” and “concurrently administering” as used herein includes administering an antagonist of FLIP activity and another disease modifying anti-rheumatoid drug in admixture, such as, for example, in a pharmaceutical composition or in solution, or as separate compounds, such as, for example, separate pharmaceutical compositions or solutions administered consecutively, simultaneously, or at different times but not so distant in time such that the antagonist of FLIP activity and other disease modifying anti-rheumatoid drug cannot interact.

Regardless of the route of administration selected, the antagonist of FLIP activity and other disease modifying anti-rheumatoid drug are formulated into pharmaceutically acceptable unit dosage forms by conventional methods known to the pharmaceutical art. An effective but nontoxic quantity of the antagonist of FLIP activity and other disease modifying anti-rheumatoid drug are employed in the treatment. The antagonist of FLIP activity and other disease modifying anti-rheumatoid drug may be concurrently administered enterally and/or parenterally in admixture or separately. Parenteral administration includes subcutaneous, intramuscular, intradermal, intravenous, injection directly into the joint and other administrative methods known in the art. Enteral administration includes tablets, sustained release tablets, enteric coated tablets, capsules, sustained release capsules, enteric coated capsules, pills, powders, granules, solutions, and the like.

H. Pharmaceutical Compositions

An aspect of the invention provides methods of manufacturing a drug useful for treating rheumatoid arthritis in a warm-blooded animal. The drug is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the invention are found by reference to the 18^{th} or 19^{th} Edition of Remington's Pharmaceutical. Sciences, Published by the Mack Publishing Co., Easton, Pa. 18040. The pertinent portions are incorporated herein by reference.

Unit doses or multiple dose forms are contemplated, each offering advantages in certain clinical settings. The unit dose would contain a predetermined quantity of an antagonist of FLIP activity calculated to produce the desired effect(s) in the setting of treating rheumatoid arthritis. The multiple dose form may be particularly useful when multiples of single doses, or fractional doses, are required to achieve the desired ends. Either of these dosing forms may have specifications that are dictated by or directly dependent upon the unique characteristic of the particular compound, the particular therapeutic effect to be achieved, and any limitations inherent in the art of preparing the particular compound for treatment of cancer.

A unit dose will contain a therapeutically effective amount sufficient to treat rheumatoid arthritis in a subject and may contain from about 1.0 to 1000 mg of compound, for example about 50 to 500 mg.

In a preferred embodiment, the drug of the invention is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The drug of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The compound will preferably be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition.

The form may deliver a compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. Preferred compositions according to the current invention are prepared so that an oral dosage unit form contains between about 5.0 to about 50% by weight (%w) in dosage units weighing between 5 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

The drug of the invention may be administered parenterally, e.g., intravenously, intramuscularly, intravenously, subcutaneously, or intraperitoneally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In a specific embodiment, it may be desirable to administer the compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.)

In yet another embodiment, the composition can be delivered in a controlled release, or sustained release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used in a controlled release system (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target (e.g., the brain, kidney, stomach, pancreas, and lung), thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the drug of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

### IV. EXAMPLES

The following examples are provided as a guide for a practitioner of ordinary skill in the art. The examples should not be construed as limiting the invention, as the examples merely provide specific methodology useful in understanding and practicing an embodiment of the invention.

### A. Example 1

### FLIP Expression

Mononuclear cells (MNC) are isolated from synovial fluid (SF) from RA patients by Histopaque (Sigma Chemical Co.) gradient centrifugation. Isolated RA synovial tissue MNC are differentiated into macrophages in 20% FBS/ RPMI/1 μg/ml polymyxin B sulfate (Sigma). MNC and macrophages are blocked for 1 hour at room temperature in 50% human serum. Following blocking, MNC are stained with phycoerytherin (PE)-conjugated anti-CD14 (Beckman, Coulter, Miami, Fla.) or control PE-labeled IgM. The CD14-labeled MNC are fixed in 4% neutral buffered formalin, permeabilized with 0.1% Nonidet P-40 (NP-40), blocked overnight at 4° C. in 90% goat serum, and incubated at 4° C. for 3-4 hours with rabbit anti-FLIP antibody of control rabbit IgG. Cells are then incubated with FITC-labeled goat anti-rabbit antibody (Jackson ImmunoResearch) at 4° C. for 1-2 hours. FLIP expression is determined in the CD14-positive MNC by flow cytometry, and intracellular FLIP is quantified by mean fluorescence intensity.

### B. Example 2

### Inhibition of FLIP Activity using Antisense Oligonucleotides

MNC and macrophages from RA synovial fluid are incubated for 24h with FITC-labeled antisense phosphorothioate oligodeoxynucleotides (10-20 μM) comprising the FLIP initiation codon (5′-GACTTCAGCAGACATCCTAC-3′) (SEQ ID NO: 2). A nonsense oligonucleotides is used as negative control (for example; 5′-TGGATCCGACATGTCAGA-3′) (SEQ ID NO: 3). Uptake of the FITC-labeled oligonucleotides are measured by flow cytometry on 70% ETOH fixed cells. A 80-90% transfection efficiency is expected. A general caspase inhibitor (e.g., 20 μM zVAD.fmk) is used as negative control in all apoptosis assays. As a positive control of macrophage apoptosis, the cells are treated with 50 μM of the phosphatidylinositol 3-kinase inhibitor LY294002 for 24 h.

### C. Example 3

### Western Blot Analysis of FLIP Expression

Whole-cell extracts are prepared from synovial MNC and macrophages by lysis in 0.1% NP-40 lysis buffer. 25 to 50 μg of extract are analyzed by SDS-PAGE on 12.5% polyacrylamide gels, and transferred to ImmobilonP (Millipore) by semidry blotting. Filters are blocked for 1 h at room temperature in PBS/0.2%Tween-20/5% nonfat dry milk. Filters are blotted with rabbit anti-FLIP antiserum or monoclonal anti-FLIP antibodies clone Dave-2 or clone NF6 (Axorra LLC, San Diego, Calif.), at 4° C. in PBS/0.2% Tween-20/2% nonfat dry milk. Filters are washed inPBS/0.2% Tween 20/2% nonfat dry milk and incubated with donkey anti-rabbit or anti-mouse secondary antibody (1:2,000 dilution) conjugated to horseradish peroxidase (Amersham PharmaciaBiotech). Visualization of the immunocomplex is performed using Enhanced Chemiluminescence Plus kit (AmershamPharmacia Biotech).

### D. Example 4

### Caspase-8 Cleavage

Apoptosis is induced in synovial MNC and macrophages by incubating the cells for 24 h with recombinant TNF (10 ng/ml), or 1 μg/ml anti-Fas, anti-TNF-R1 or anti-TRAIL receptors antibodies. 2×10⁶ monocytes are centrifuged at 400×G for minutes, the supernatant is discarded and the cells are lysed in Tris buffered saline containing detergent. The cells are incubated on ice for 10 minutes and then centrifuged in a microcentrifuge at maximum speed for 10 minutes at 4° C. 50 μl of lysed cell supernatant is combined with 50 PI reaction buffer [Tris buffered saline with detergent including 10 mM dithiothreitol, DTT] and 1 μl of the test compound in DMSO. The mixture is incubated for 30 minutes on ice. In addition, one sample containing 50 μl lysed cell supernatant, 50 μl reaction buffer and 1 μl DMSO is incubated for 30 minutes on ice. After incubation, 5 μl of 4 mM IETD-pNA (200 μM final conc.) is added to each reaction mixture. The samples are then incubated at 37° C. for one hour. Cleavage is detected by calorimetric detection of free p-nitroanilide (pNA) at 405 nm.

### E. Example 5

### Apoptosis Activation and Annexin V Assay

Isolated RA synovial fluid MNC and macrophages are incubated with 1μg/ml of anti-Fas antibody (clone CH11; Beckman Coulter) or irrelevant IgM monoclonal antibody control for 24 hours. Cells are washed twice with cold PBS and then resuspended in 10 mM HEPES, pH 7.4; 140 mM NaCl; 2.5 mM CaCl₂ at a concentration of ˜1×10⁶ cells/ml. 100 μl of the solution (˜1×10⁵ cells) is transferred to a 5 ml culture tube. 5 μl of 2.5 μg Annexin V-phycoerythrin and 2.5 μg vital dye 7-AAD are added to each tube, gently mixed and incubated at room temperature in the dark for 15 minutes. 400 μl phosphate buffered saline (PBS) is added to each tube and the cells are analyzed by cell cytometry as soon as possible (within one hour). The percentage of apoptotic cells is measured by the percentage of Annexin V positive cells.

### F. Example 6

### TUNEL Assay

Apoptosis is induced in synovial MNC and macrophages by incubating the cells for 24 h with recombinant TNF (10 ng/ml), or 1 μg/ml anti-Fas, anti-TNF-R1 or anti-TRAIL receptors antibodies 1-2×10⁶ monocytes are centrifuged at 400×G for minutes, the supernatant is discarded and the cells are resuspended in 0.5 ml phosphate buffered saline (PBS). The cells are fixed by adding the cell suspension to 5 ml of 1% (w/v) paraformaldehyde in PBS, placing it on ice for 15 min, washing the cells twice in PBS twice, and finally combining the cells suspended in 0.5 ml PBS with 5 ml ice-cold 70% (v/v) ethanol. The cells stand for a minimum of 30 minutes on ice or in the freezer before proceeding to the staining step.

The tubes are swirled to resuspend the cells and 1.0 ml aliquots of the cell suspensions (˜2-4×10⁵ cells/ml) are removed and placed in 12×75 mm centrifuge tubes. The cell suspensions are centrifuged for 5 min at 300×g and the 70% (v/v) ethanol removed by aspiration. The cells are washed twice by centrifugation and resuspension in PBS plus 0.05% sodium azide, pelleted and then resuspended in 50 μl Staining Solution (TdT enzyme/FITC-dUTP in cacodylate buffered saline). The cells are incubated at 37° C. for at least one hour. The staining is stopped by the addition of 1.0 ml PBS pus 0.05% sodium azide. The cells are pelleted by centrifugation at 300×g for 5 min, resuspended in PBS pus 0.05% sodium azide, and the repelleted. The supernatant is removed by aspiration and the pellet is incubated for 30 minutes at room temperature in the dark. The cells are analyzed by flow cytometry.

### G. Example 7

### Propidium Iodide Staining

9-day adherent synovial fluid macrophages are incubated with anti-Fas antibody or control IgM in the presence and absence of the test compound for 24 hours. Cultures are then harvested by 0.02% EDTA, fixing overnight in 70% ethanol, stained with propidium iodide (Roche Molecular Biochemicals, Indianapolis, Ind.), and the subdiploid peak, immediately next to the G₀/G₁ peak (2N), is determined by flow cytometry. It may be necessary to exclude objects with minimal light scatter, possibly representing debris, which would artificially increase the estimate of the subdiploid population. Typically, the percentage of apoptotic synovial macrophages (subdiploid population) increase from 2-5% in absence of FLIP antagonist to 35-40% when FLIP activity is completely suppressed.

### H. Example 8

### Anti-histone Sandwich Assay

Apoptosis is induced by incubating 10⁴ synovial MNC or macrophages with 1 μg/ml anti-Fas antibody (CH11) or TNF-α (10 ng/ml) for 24 h. After the incubation, the cells are pelleted by centrifugation and the supernatant (containing DNA from necrotic cells that leaked through the membrane during incubation) is discarded. The cells are resuspended in Lysis Buffer and incubated 30 min at room temperature. After lysis, cell nuclei and unfragmented DNA are pelleted by centrifugation at 20 000×g for 10 min.

An aliquot of the supernatant (i.e., cytoplasmic fraction) is transferred to anti-histone antibody well of a microtiter plate. The complexes are bound to the plate via streptavidin-biotin interaction. The immobilized antibody-DNA-antibody complexes are washed three times to remove any components that are not immunoreactive. The bound complexes are detected with anti-DNA (peroxidase-conjugated) monoclonal antibodies revealed by a peroxidase substrate and amount of colored product (and thus, of immobilized antibody-histone complexes) is determined spectrophotometrically. The quantitative calorimetric measurement of the DNA-histone complex is proportional to the total amount of apoptotic cells present in the cell population tested.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of alleviating at least one symptom of rheumatoid arthritis comprising administering a therapeutically effective amount of an antagonist of FLIP activity to a patient having rheumatoid arthritis, wherein the antagonist decreases FLIP activity by at least 25%.

2. The method of claim 1, wherein the antagonist decreases FLIP activity by at least 50%.

3. The method of claim 2, wherein the antagonist decreases FLIP activity by at least 70%.

4. The method of claim 3, wherein the antagonist decreases FLIP activity by at least 95%.

5. The method of claim 1, wherein the antagonist of FLIP activity is a protein.

6. The method of claim 5, wherein the protein is oxidized low-density lipoprotein, ectopic-p53, IFN-β, PPAR ligand, E1A, or hemin.

7. The method of claim 1, wherein the antagonist of FLIP activity is a nucleic acid.

8. The method of claim 7, wherein the nucleic acid is an antisense inhibitor.

9. The method of claim 8, wherein the antisense inhibitor comprises the sequence, 5′-GACTTCAGCAGACATCCTAC-3′ (SEQ ID NO: 2).

10. The method of claim 1, wherein the antagonist of FLIP activity is a small molecule.

11. The method of claim 10, wherein the small molecule is selected from the group consisting of cyclohexamide, actinomycin D, 5-fluorouracil, doxorubicin, cisplatin, sodium butyrate, bisindolylmaleimides, H7, calphostin C, chelerythrine chloride, CDDO (triterpenoid 2-cyano-3,12-dioxoolean-1,9-dien-28-oic acid) and PS-341.

12. A method of decreasing density of synovial cells in a joint comprising administering a therapeutically effective amount of an antagonist of FLIP activity to a patient having a condition associated with abnormally increased synovial cell density, wherein antagonist decreases FLIP activity by at least 25%.

13. The method of claim 12, wherein the antagonist decreases FLIP activity by at least 50%.

14. The method of claim 13, wherein the antagonist decreases FLIP activity by at least 70%.

15. The method of claim 14, wherein the antagonist decreases FLIP activity by at least 95%.

16. The method of claim 12, wherein the antagonist of FLIP activity is a protein.

17. The method of claim 16, wherein the protein is oxidized low-density lipoprotein, ectopic-p53, IFN-β, PPAR ligand, E1A, or hemin.

18. The method of claim 12, wherein the antagonist of FLIP activity is a nucleic acid.

19. The method of claim 18, wherein the nucleic acid is an antisense inhibitor.

20. The method of claim 19, wherein the antisense inhibitor comprises the sequence, 5′-GACTTCAGCAGACATCCTAC-3′ (SEQ ID NO: 2).

21. The method of claim 12, wherein the antagonist of FLIP activity is a small molecule.

22. The method of claim 21, wherein the small molecule is selected from the group consisting of cyclohexamide, actinomycin D, 5-fluorouracil, doxorubicin, cisplatin, sodium butyrate, bisindolylmaleimides, H7, calphostin C, chelerythrine chloride, CDDO (triterpenoid 2-cyano-3,12-dioxoolean-1,9-dien-28-oic acid) and PS-341.

23. A method of decreasing cartilage degradation in a joint comprising administering a therapeutically-effective amount of an antagonist of FLIP activity to a patient having a condition associated with an abnormally high rate of cartilage degradation, wherein the antagonist decreases FLIP activity by at least 25%.

24. The method of claim 23, wherein the antagonist decreases FLIP activity by at least 50%.

25. The method of claim 24, wherein the antagonist decreases FLIP activity by at least 70%.

26. The method of claim 25, wherein the antagonist decreases FLIP activity by at least 95%.

27. The method of claim 23, wherein the antagonist of FLIP activity is a protein.

28. The method of claim 27, wherein the protein is oxidized low-density lipoprotein, ectopic-p53, IFN-β, PPAR ligand, E1A, or hemin.

29. The method of claim 23, wherein the antagonist of FLIP activity is a nucleic acid.

30. The method of claim 29, wherein the nucleic acid is an antisense inhibitor.

31. The method of claim 30, wherein the antisense inhibitor comprises the sequence, 5′-GACTTCAGCAGACATCCTAC-3′ (SEQ ID NO: 2).

32. The method of claim 23, wherein the antagonist of FLIP activity is a small molecule.

33. The method of claim 32, wherein the small molecule is selected from the group consisting of cyclohexamide, actinomycin D, 5-fluorouracil, doxorubicin, cisplatin, sodium butyrate, bisindolylmaleimides, H7, calphostin C, chelerythrine chloride, CDDO (triterpenoid 2-cyano-3,12-dioxoolean-1,9-dien-28-oic acid) and PS-341.

34. A method of manufacturing a drug for use in the treatment of rheumatoid arthritis comprising:
(a) identifying a compound as useful in the treatment of rheumatoid arthritis by:
(i) comparing an amount of FLIP activity in the presence of the compound with an amount FLIP activity in the absence of the compound; and
(ii) identifying the compound as useful in the treatment of rheumatoid arthritis when the amount of FLIP activity in the presence of the compound is at least 25% lower than the amount of FLIP activity in the absence of the compound; and
(b) formulating said compound for human consumption.

35. The method of claim 34, wherein the compound is identified as useful in the treatment of rheumatoid arthritis when the amount of FLIP activity in the presence of the compound is at least 50% lower than the amount of FLIP activity in the absence of the compound.

36. The method of claim 35, wherein the compound is identified as useful in the treatment of rheumatoid arthritis when the amount of FLIP activity in the presence of the compound is at least 70% lower than the amount of FLIP activity in the absence of the compound.

37. The method of claim 36, wherein the compound is identified as useful in the treatment of rheumatoid arthritis when the amount of FLIP activity in the presence of the compound is at least 95% lower than the amount of FLIP activity in the absence of the compound.

38. The method of claim 34, wherein the amount of FLIP activity is measured by a process comprising the steps of:
(1) adding a caspase-8 substrate to a cell lysate in the presence or absence of the compound; and
(2) measuring the amount of caspase-8 substrate cleaved wherein the compound is identified as useful in the treatment of rheumatoid arthritis when the amount of caspase-8 substrate cleaved in the presence of the compound is at least 50% greater than the amount of caspase-8 substrate cleaved in the absence of the compound.

39. The method of claim 38, wherein the compound is identified as useful in the treatment of rheumatoid arthritis when the amount of caspase-8 substrate cleaved in the presence of the compound is at least 100% greater than the amount of caspase-8 substrate cleaved in the absence of the compound.

40. The method of claim 39, wherein the compound is identified as useful in the treatment of rheumatoid arthritis when the amount of caspase-8 substrate cleaved in the presence of the compound is at least 200% greater than the amount of caspase-8 substrate cleaved in the absence of the compound.

41. The method of claim 38, wherein the caspase-8 substrate is IETD (SEQ ID NO: 1) conjugated to p-nitroanilide and the amount cleaved is measured calorimetrically.

42. The method of claim 38, wherein the caspase-8 substrate is IETD (SEQ ID NO: 1) conjugated to a fluorescent marker.

43. The method of claim 38, further comprising the step of:
exposing cells to an inducer of apoptosis in the presence or absence the compound prior to lysing the cells to produce a cell lysate.

44. The method of claim 43, wherein the inducer of apoptosis is selected from the group consisting of Fas ligand, TRAIL, TNF-α or an anti-death receptor antibody.

45. The method of claim 44, wherein the anti-death receptor antibody is an anti-TNF-R1 antibody, an anti-Fas antibody, an anti-TRAIL-R antibody or an anti-DR6 antibody.

46. The method of claim 34, wherein the amount of FLIP activity is measured by measuring the amount of FLIP protein expressed in a population of cells in the presence or absence of the compound.

47. A method of manufacturing a drug for use in the treatment of rheumatoid arthritis comprising:
(a) identifying a compound as useful in the treatment of rheumatoid arthritis by:
(i) comparing an amount of macrophage apoptosis in the presence of the compound with an amount macrophage apoptosis in the absence of the compound; and
(ii) identifying the compound as useful in the treatment of rheumatoid arthritis when the amount of macrophage apoptosis in the presence of the compound is at least 50% greater than the amount of macrophage apoptosis in the absence of the compound; and
(b) formulating said compound for human consumption.

48. The method of claim 47, wherein the compound is identified as useful in the treatment of rheumatoid arthritis when the amount of macrophage apoptosis in the presence of the compound is at least 100% greater than the amount of macrophage apoptosis in the absence of the compound.

49. The method of claim 48, wherein the compound is identified as useful in the treatment of rheumatoid arthritis when the amount of macrophage apoptosis in the presence of the compound is at least 200% greater than the amount of macrophage apoptosis in the absence of the compound.

50. The method of claim 47, wherein the compound identified as useful in the treatment of rheumatoid arthritis decreases FLIP activity by at least 25%.

51. The method of claim 50, wherein the compound identified as useful in the treatment of rheumatoid arthritis decreases FLIP activity by at least 50%.

52. The method of claim 51, wherein the compound identified as useful in the treatment of rheumatoid arthritis decreases FLIP activity by at least 70%.

53. The method of claim 52, wherein the compound identified as useful in the treatment of rheumatoid arthritis decreases FLIP activity by at least 95%.

54. The method of claim 47, wherein the amount of macrophage apoptosis is measured by a process comprising the steps of:
(1) exposing a population of cells to an inducer of apoptosis in the presence or absence of the compound; and
(2) measuring the percentage of cells in the population having DNA fragmentation
wherein the percentage of cells having DNA fragmentation represents the amount of macrophage apoptosis.

55. The method of claim 54, wherein the inducer of apoptosis is selected from the group consisting of Fas ligand, TRAIL, TNF-α or an anti-death receptor antibody.

56. The method of claim 55, wherein the anti-death receptor antibody is an anti-TNF-R1 antibody, an anti-Fas antibody, an anti-TRAIL-R antibody or an anti-DR6 antibody.

57. The method of claim 54, wherein the percentage of cells having DNA fragmentation is measured by FACS analysis of propidium uptake of cells.

58. The method of claim 54, wherein the percentage of cells having DNA fragmentation is measured by TUNEL assay.

59. The method of claim 47, wherein the amount of macrophage apoptosis is measured by a process comprising the steps of:
(1) exposing a population of cells to an inducer of apoptosis in the presence or absence of the compound; and
(2) measuring a percentage of cells in the population expressing phosphatidylserine on the extracellular surface of the cell membrane
wherein the percentage of cells expressing phosphatidylserine on the extracellular surface of the cell membrane represents the amount of macrophage apoptosis.

60. The method of claim 59, wherein the inducer of apoptosis is selected from the group consisting of Fas ligand, TRAIL, TNF-α or an anti-death receptor antibody.

61. The method of claim 60, wherein the anti-death receptor antibody is an anti-TNF-R1 antibody, an anti-Fas antibody, an anti-TRAIL-R antibody or an anti-DR6 antibody.

62. The method of claim 59, wherein the percentage of cells expressing phosphatidylserine present on the extracellular surface of the cytoplasmic membrane is measured by binding of annexin V to the phosphatidylserine.

63. The method of claim 62, wherein the annexin V is conjugated to a fluorescent marker.

64. A method of screening a collection of compounds for a compound useful in the treatment of rheumatoid arthritis comprising:
(a) comparing an amount of FLIP activity in the presence of the compound with an amount FLIP activity in the absence of the compound; and
(b) selecting the compound as useful in the treatment of rheumatoid arthritis when the amount of FLIP activity in the presence of the compound is at least 25% lower than the amount of FLIP activity in the absence of the compound.

65. The method of claim 64, wherein the compound is identified as useful in the treatment of rheumatoid arthritis when the amount of FLIP activity in the presence of the compound is at least 50% lower than the amount of FLIP activity in the absence of the compound.

66. The method of claim 65, wherein the compound is identified as useful in the treatment of rheumatoid arthritis when the amount of FLIP activity in the presence of the compound is at least 70% lower than the amount of FLIP activity in the absence of the compound.

67. The method of claim 66, wherein the compound is identified as useful in the treatment of rheumatoid arthritis when the amount of FLIP activity in the presence of the compound is at least 95% lower than the amount of FLIP activity in the absence of the compound.

68. The method of claim 64, wherein the amount of FLIP activity is measured by a process comprising the steps of:
(1) adding a caspase-8 substrate to a cell lysate in the presence or absence of the compound; and
(2) measuring the amount of caspase-8 substrate cleaved
wherein the compound is selected as useful in the treatment of rheumatoid arthritis when the amount of caspase-8 substrate cleaved in the presence of the compound is at least 50% greater than the amount of caspase-8 substrate cleaved in the absence of the compound.

69. The method of claim 68, wherein the compound is selected as useful in the treatment of rheumatoid arthritis when the amount of caspase-8 substrate cleaved in the presence of the compound is at least 100% greater than the amount of caspase-8 substrate cleaved in the absence of the compound.

70. The method of claim 69, wherein the compound is selected as useful in the treatment of rheumatoid arthritis when the amount of caspase-8 substrate cleaved in the presence of the compound is at least 200% greater than the amount of caspase-8 substrate cleaved in the absence of the compound.

71. The method of claim 68, wherein the caspase-8 substrate is IETD (SEQ ID NO: 1) conjugated to pNA and the amount cleaved is measured calorimetrically.

72. The method of claim 68, wherein the caspase-8 substrate is IETD (SEQ ID NO: 1) conjugated to a fluorescent marker.

73. The method of claim 68, further comprising the step of:
exposing cells to an inducer of apoptosis in the presence or absence the compound prior to lysing the cells to produce a cell lysate.

74. The method of claim 73, wherein the inducer of apoptosis is selected from the group consisting of Fas ligand, TRAIL, TNF-α or an anti-death receptor antibody.

75. The method of claim 74, wherein the anti-death receptor antibody is an anti-TNF-R1 antibody, an anti-Fas antibody, an anti-TRAIL-R antibody or an anti-DR6 antibody.

76. The method of claim 64, wherein the amount of FLIP activity is measured by measuring the amount of FLIP protein expressed in a population of cells in the presence or absence of the compound.

77. The method of claim 64, further comprising repeating steps (a) and (b) for each compound of the collection, wherein at least one compound of the collection is selected as useful in the treatment of rheumatoid arthritis.

78. A method of screening a collection of compounds for a compound useful in the treatment of rheumatoid arthritis comprising:
(a) comparing an amount of macrophage apoptosis in the presence of the compound with an amount macrophage apoptosis in the absence of the compound ; and
(b) selecting the compound as useful in the treatment of rheumatoid arthritis when the amount of macrophage apoptosis in the presence of the compound is at least 50% greater than the amount of macrophage apoptosis in the absence of the compound.

79. The method of claim 78, wherein the compound is selected as useful in the treatment of rheumatoid arthritis when the amount of macrophage apoptosis in the presence of the compound is at least 100% greater than the amount of macrophage apoptosis in the absence of the compound.

80. The method of claim 79, wherein the compound is selected as useful in the treatment of rheumatoid arthritis when the amount of macrophage apoptosis in the presence of the compound is at least 200% greater than the amount of macrophage apoptosis in the absence of the compound.

81. The method of claim 78, wherein the compound selected as useful in the treatment of rheumatoid arthritis decreases FLIP activity by at least 25%.

82. The method of claim 81, wherein the compound selected as useful in the treatment of rheumatoid arthritis decreases FLIP activity by at least 50%.

83. The method of claim 82, wherein the compound selected as useful in the treatment of rheumatoid arthritis decreases FLIP activity by at least 70%.

84. The method of claim 83, wherein the compound selected as useful in the treatment of rheumatoid arthritis decreases FLIP activity by at least 95%.

85. The method of claim 78, further comprising repeating steps (a) and (b) for each compound of the collection, wherein at least one compound of the collection is selected as useful in the treatment of rheumatoid arthritis.

86. The method of claim 78, wherein the amount of macrophage apoptosis is measured by a process comprising the steps of:
(1) exposing a population of cells to an inducer of apoptosis in the presence or absence of the compound; and
(2) measuring the percentage of cells in the population having DNA fragmentation
wherein the percentage of cells having DNA fragmentation represents the amount of macrophage apoptosis.

87. The method of claim 86, wherein the inducer of apoptosis is selected from the group consisting of Fas ligand, TRAIL, TNF-α or an anti-death receptor antibody.

88. The method of claim 87, wherein the anti-death receptor antibody is an anti-TNF-R1 antibody, an anti-Fas antibody, an anti-TRAIL-R antibody or an anti-DR6 antibody.

89. The method of claim 86, wherein the percentage of cells having DNA fragmentation is measured by FACS analysis of propidium uptake of cells.

90. The method of claim 86, wherein the percentage of cells having DNA fragmentation is measured by TUNEL assay.

91. The method of claim 78, wherein the amount of macrophage apoptosis is measured by a process comprising the steps of:
(1) exposing a population of cells to an inducer of apoptosis in the presence or absence of the compound; and
(2) measuring a percentage of cells in the population expressing phosphatidylserine on the extracellular surface of the cell membrane
wherein the percentage of cells expressing phosphatidylserine on the extracellular surface of the cell membrane represents the amount of macrophage apoptosis.

92. The method of claim 91, wherein the inducer of apoptosis is selected from the group consisting of Fas ligand, TRAIL, TNF-α or an anti-death receptor antibody.

93. The method of claim 92, wherein the anti-death receptor antibody is an anti-TNF-R1 antibody, an anti-Fas antibody, an anti-TRAIL-R antibody or an anti-DR6 antibody.

94. The method of claim 91, wherein the percentage of cells expressing phosphatidylserine present on the extracellular surface of the cytoplasmic membrane is measured by binding of annexin V to the phosphatidylserine.

95. The method of claim 94, wherein the annexin V is conjugated to a fluorescent marker.

96. A method of alleviating at least one symptom of rheumatoid arthritis, comprising administering an antagonist of FLIP activity and a disease-modifying anti-rheumatic drug to a patient having rheumatoid arthritis, wherein the anti-rheumatic drug is selected from the group of methotrexate, an interleukin-1 receptor antagonist and a steroid.

97. The method of claim 96, wherein the patient is a methotrexate resistant patient and the anti-rheumatic drug is methotrexate or an interleukin-1 receptor antagonist.

98. The method of claim 96, wherein the patient is a TNF-α blockade resistant patient and the anti-rheumatic drug is an interleukin-1 receptor antagonist or a steroid.

99. The method of claim 98, wherein the a TNF-α blockade hyperplasia nonresponder
